(19) United States
(12) Patent Application Publication
(10) Pub. No.: US 2016/0067271 A1
(43) Pub. Date: Mar. 10, 2016

(54) Methods And Compositions For Preventing Or Treating Obesity

(71) Applicant: Northeastern University, Boston, MA (US)

(72) Inventors: Michail V. SITKOVSKY, Boston, MA (US); Akio OHTA, Newton, MA (US)

(21) Appl. No.: 14/853,414

(22) Filed: Sep. 14, 2015

Related U.S. Application Data
(63) Continuation of application No. 13/819,229, filed on Aug. 12, 2013, now abandoned.
(60) Provisional application No. 61/377,298, filed on Aug. 26, 2010.

(51) Int. Cl. A61K 31/7076; A61K 31/365; A61K 38/22; A61K 45/06

(52) U.S. Cl. CPC A61K 31/7076; A61K 45/06; A61K 31/365; A61K 38/2264

(57) ABSTRACT

The invention includes methods of treating, preventing, or limiting obesity or weight gain, or reducing or suppressing appetite, by the administration of A2A adenosine receptor pathway agonists. The A2AR pathway agonists may be administered in conjunction with a therapeutic agent having a side effect of weight gain, in order to prevent or limit that weight gain. In some instances, the A2AR pathway agonist is administered as a sleeping pill, and in other instances the A2AR pathway agonist is administered in a non-drowsy formulation.

### CROSS-REFERENCE TO RELATED APPLICATION

This application claims the benefit of U.S. Provisional Application No. 61/377,298, filed Aug. 26, 2010. The entire teachings of the referenced application are expressly incorporated herein by reference.

### BACKGROUND

Obesity is a major health concern in Western societies. It is estimated that about 97 million adults in the United States are overweight or obese. The medical problems associated with obesity, which can be serious and life-threatening, include hypertension; type 2 diabetes mellitus; elevated plasma insulin concentrations; insulin resistance; dyslipidemias; hyperlipidemia; endometrial, breast, prostate and colon cancer; osteoarthritis; respiratory complications, such as obstructive sleep apnea; cholelithiasis; gallstones; arteriosclerosis; heart disease; abnormal heart rhythms; and heart arrhythmias (Kopelman, P. G., Nature 404, 635-643 (2000)). Obesity is further associated with premature death and with a significant increase in mortality and morbidity from stroke, myocardial infarction, congestive heart failure, coronary heart disease, and sudden death. Obesity also exacerbates many health problems, both independently and in association with other diseases.

Adenosine modulates diverse physiological functions including modulation of adenylate cyclase, induction of sedation, vasodilatation, suppression of cardiac rate and contractility, inhibition of platelet aggregability, stimulation of gluconeogenesis and inhibition of lipolysis. In addition to its effects on adenylate cyclase, adenosine opens potassium channels, reduces flux through calcium channels, and inhibits or stimulates phosphoinositide turnover through receptor-mediated mechanisms. Based on biochemical and pharmacological criteria, four subtypes of adenosine receptors have been differentiated: A2a, A2b, A1, and A3. A1 and A3 inhibit, and A2a and A2b stimulate, adenylate cyclase, respectively. The cDNAs that encode the A1, A2, and A3 adenosine receptors have been cloned. Molecular cloning of the adenosine receptors has revealed that they belong to the superfamily of G-protein coupled receptors.

There exists a need in the art for new methods of treating and preventing obesity. Others have suggested that adenosine receptor 2A (A2AR) antagonists may be used as anti-obesity therapy (see U.S. Pat. No. 6,664,252). Applicants have surprisingly shown that the opposite is true: A2AR pathway agonists are effective in preventing or treating obesity.

### SUMMARY

In certain aspects, this disclosure provides a method of reducing appetite, comprising administering a therapeutically effective amount of an A2AR pathway agonist to an animal in need thereof. In certain embodiments, the disclosures herein provide a method of preventing or limiting weight gain and/or reducing appetite, comprising administering to an animal a therapeutically effective amount of an A2AR pathway agonist sufficient to reduce weight gain and/or reduce appetite under conditions where the animal, in the absence of said agonist, would be susceptible to weight gain and/or increased appetite. Herein is also provided a method of preventing or limiting weight gain and/or reducing appetite, consisting of administering to an animal a therapeutically effective amount of an A2AR pathway agonist sufficient to reduce weight gain under conditions where the animal, in the absence of said agonist, would be susceptible to weight gain.

In some instances, the A2AR agonist is a drug that increases extracellular adenosine levels such as an adenosine kinase inhibitor or an inhibitor of an adenosine-degrading enzyme, such as adenosine deaminase (ADA). In some instances, the weight gain comprises an increase in fat. The fat may be, for example, visceral fat or subcutaneous fat. In certain embodiments, the animal is not conjointly being treated with an antihistamine, a protein tyrosine phosphatase inhibitor, a COX-2 inhibitor, a FAAH inhibitor, a CRTH2 modulator, or an anti-cholinergic agent.

This application also provides, inter alia, a method of treating obesity comprising administering a therapeutically effective amount of an A2AR pathway agonist to an animal in need thereof. Also disclosed is a method of treating obesity consisting of administering a therapeutically effective amount of an A2AR pathway agonist to an animal in need thereof. In certain embodiments, the animal is not conjointly being treated with an antihistamine, a protein tyrosine phosphatase inhibitor, a COX-2 inhibitor, a FAAH inhibitor, a CRTH2 modulator, an anti-cholinergic agent, an adrenergic receptor antagonist, or a kinase inhibitor.

Also provided by the instant disclosure is a method of preventing or limiting weight gain induced by a therapeutic agent that induces weight gain, comprising administering a therapeutically effective amount of an A2AR pathway agonist to an animal that is being treated with the therapeutic agent. In addition, this application provides a method of preventing or limiting weight gain induced by a therapeutic agent that induces weight gain, consisting of administering a therapeutically effective amount of an A2AR pathway agonist to an animal that is being treated with the therapeutic agent.

In some embodiments, the weight gain that is prevented or limited comprises an increase in fat. The therapeutic agent that causes weight gain may be, for example, a diabetes therapeutic or an antidepressant. The diabetes therapeutic may be at least one of: a sulfonylurea, a thiazolidinedione, a meglitinide, nateglinide, repaglinide, or insulin. Furthermore, the antidepressant may be at least one of: a tricyclic antidepressant, an irreversible monoamine oxidase inhibitor (MAOI), a selective scrotonin reuptake inhibitor (SSRI), bupropion, paroxetine, or mirtazapine.

In some embodiments, this application discloses a method of treating, preventing, or limiting weight gain, comprising conjointly administering to an animal in need thereof: (a) a therapeutically effective amount of an A2AR pathway agonist, and (b) one or more additional therapy, wherein the additional therapy treats, limits or prevents obesity.

In certain embodiments, the animal is not being conjointly treated with an antihistamine, a protein tyrosine phosphatase inhibitor, a COX-2 inhibitor, a FAAH inhibitor, a CRTH2 modulator, an anti-cholinergic agent, an adrenergic receptor antagonist, or a kinase inhibitor.

In certain aspects, the disclosures herein provide a method of treating, preventing, or limiting weight gain, consisting of conjointly administering to an animal in need thereof: (a) a therapeutically effective amount of an A2AR pathway agonist, and (b) one or more additional therapy, wherein the additional therapy treats, limits, or prevents obesity.

The weight gain, in certain aspects, comprises an increase in fat.

The additional therapy that treats, limits, or prevents obesity may be the administration of a body weight management agent. For instance, the body weight management agent may be an appetite suppressant. In certain aspects, the appetite suppressant is selected from: aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, leptin, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex, and sibutramine. Alternatively, the body weight management agent may be a fat absorption inhibitor such as orlistat. The body weight management agent may also be a fat mobilization agent such as leptin, a leptin analog, or a leptin mimetic. In other embodiments, the additional therapy that treats, limits, or prevents obesity is a diet regimen, exercise regimen, or surgery. The surgery may be gastric bypass surgery, a restriction operation, or liposuction.

Additionally, this application provides a method of inducing satiety in an animal, comprising administering an effective amount of an A2AR pathway agonist to an animal in need thereof. In some embodiments, the animal is suffering from bulimia.

The disclosures herein additionally provide a method of treating bulimia in an animal, comprising administering a therapeutically effective amount of an A2AR pathway agonist to an animal in need thereof.

Furthermore, herein is disclosed a method of reducing appetite, comprising administering an effective amount of an A2AR pathway agonist to an animal in need thereof. Inter alia, this application provides a method of reducing appetite, comprising administering to an animal an effective amount of an A2AR pathway agonist sufficient to reduce appetite below the level of appetite that the animal would experience in the absence of said agonist.

This disclosure also provides a method of inducing satiety, comprising or consisting of administering to an animal an effective amount of an A2AR pathway agonist sufficient to increase satiety above the level of appetite that the animal would experience in the absence of said agonist. This disclosure also provides a method of inducing satiety, comprising or consisting of administering an effective amount of an A2AR pathway agonist to an animal in need thereof. Furthermore, herein is provided a method of reducing appetite, comprising or consisting of administering to an animal an effective amount of an A2AR pathway agonist to an animal in need thereof. Furthermore, herein is provided a method of reducing appetite, comprising or consisting of administering to an animal an effective amount of an A2AR pathway agonist sufficient to reduce appetite below the level of appetite that the animal would experience in the absence of said agonist.

In certain aspects, the animal is a human. In other aspects, the animal is a non-human animal. The animal may be obese. In other aspects, the animal is non-obese. In certain aspects, the animal is susceptible to obesity.

In some aspects, the A2AR pathway agonist is a specific A2AR agonist. In certain embodiments, the A2AR agonist binds A2AR. In certain embodiments, the A2AR agonist is a small molecule that binds A2AR. For example, the A2AR agonist may be APEC, ATL-146e, ATL202, ATL-313, ATL359, ATL844, ATL902, ATL908, ATL1222, ATL9844, binodenoson, CGS21680, CGS 22492C, CHA, CV-3146, CVT-3033, DMPA, GW328267X, LUF5835, MRE-0094, NECA, regadonoson, UK-371104, UK-432097, or CV1808.

In certain embodiments, the A2AR pathway agonist reduces the activity of an inhibitor of the A2AR pathway. The inhibitor of the A2AR pathway may be adenosine kinase or adenosine deaminase. The A2AR pathway agonist may be a siRNA or ribozyme that reduces the levels of the inhibitor of the A2AR pathway. The A2AR pathway agonist may also be an activator of an adenosine synthesizing enzyme such as endoNTase. Other adenosine synthesizing enzymes include CD39 and CD73. The A2AR pathway agonist may inhibit an enzyme that degrades adenosine, such as adenosine kinase or adenosine deaminase.

In some aspects, the A2AR pathway agonist is administered once nightly. In certain embodiments, the A2AR pathway agonist promotes sleep. In other embodiments, the A2AR pathway agonist does not induce drowsiness. The A2AR pathway agonist may be administered once per day, prior to sleeping. In some aspects, the animal is suffering from insomnia. In some aspects, the animal is suffering from an inflammatory disease.

This application also discloses a pharmaceutical composition comprising: (a) one or more pharmaceutically acceptable carriers, (b) a therapeutically effective amount of an A2AR pathway agonist, and (c) a therapeutic agent that causes weight gain. In some instances, the composition does not contain an antihistamine, a tyrosine phosphatase inhibitor, a COX-2 inhibitor, a FAAH inhibitor, a CRTH2 modulator, an anti-cholinergic agent, an adrenergic receptor antagonist, or a kinase inhibitor. Herein is also disclosed a pharmaceutical composition consisting of: (a) one or more pharmaceutically acceptable carriers, (b) a therapeutically effective amount of an A2AR pathway agonist, and (c) a therapeutic agent that causes weight gain.

The therapeutic agent that causes weight gain may be, for example, a diabetes therapeutic or an antidepressant. The diabetes therapeutic may be at least one of: a sulfonylurea, a thiazolidinedione, a meglitinide, nateglinide, repaglinide, or insulin. The antidepressant may be at least one of: a tricyclic antidepressant, an irreversible monoamine oxidase inhibitor (MAOI), a selective serotonin reuptake inhibitor (SSRI), bupropion, paroxetine, or mirtazapine.

In some embodiments, the disclosure contemplates a pharmaceutical composition comprising: (a) one or more pharmaceutically acceptable carriers, (b) a therapeutically effective amount of an A2AR pathway agonist, and (c) a body weight management agent. In certain embodiments, the composition does not contain an antihistamine, a tyrosine phosphatase inhibitor, a COX-2 inhibitor, a FAAH inhibitor, a CRTH2 modulator, an anti-cholinergic agent, an adrenergic receptor antagonist, or a kinase inhibitor. Furthermore, the instant application provides a pharmaceutical composition consisting of: (a) one or more pharmaceutically acceptable carriers, (b) a therapeutically effective amount of an A2AR pathway agonist, and (c) a body weight management agent.

In some embodiments, the body weight management agent is an appetite suppressant such as aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, leptin, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine. In addition, the body weight management agent may be a fat absorption inhibitor such as orlistat. Also, the body weight management agent may be a fat mobilization agent including leptin, a leptin analog, or a leptin mimetic.

In certain embodiments, the present disclosure provides a pharmaceutical composition comprising: (a) a pharmaceutically acceptable carrier, (b) a therapeutically effective amount of an A2AR pathway agonist, and (c) an agent that promotes sleep. In some instances, the composition does not contain an antihistamine, a tyrosine phosphatase inhibitor, a COX-2 inhibitor, a FAAH inhibitor, a CRTH2 modulator, an anti-cholinergic agent, an adrenergic receptor antagonist, or a kinase inhibitor. This application also discloses a pharmaceutical composition consisting of: (a) a pharmaceutically acceptable carrier, (b) a therapeutically effective amount of an A2AR pathway agonist, and (c) an agent that promotes sleep. The additional agent that promotes sleep may be a barbiturate, benzodiazepine, antidepressant, antipsychotic, herbal sedative, or nonbenzodiazepine sedative. The pharmaceutical compositions herein may be formulated for administration once daily before sleeping.

Also provided herein is a pharmaceutical composition comprising: (a) a pharmaceutically acceptable carrier, (b) a therapeutically effective amount of an A2AR pathway agonist, and (c) an agent that promotes wakefulness. In some aspects, the composition does not contain an antihistamine, a tyrosine phosphatase inhibitor, a COX-2 inhibitor, a FAAH inhibitor, a CRTH2 modulator, an anti-cholinergic agent, an adrenergic receptor antagonist, or a kinase inhibitor. Additionally provided is a pharmaceutical composition consisting of: (a) a pharmaceutically acceptable carrier, (b) a therapeutically effective amount of an A2AR pathway agonist, and (c) an agent that promotes wakefulness. In certain embodiments, the additional agent that promotes wakefulness is a phentermine, a phenethylamine, ritalin, ephedrine, an amphetamine, a mixed amphetamine salt, methylphenidate, modafinil, methamphetamine, dexamphetamine, a norepinephrine reuptake inhibitor, a dopamine reuptake inhibitor, or an ampakine.

This disclosure also provides a pharmaceutical composition comprising: (a) a pharmaceutically acceptable carrier, (b) a therapeutically effective amount of an A2AR pathway agonist, and (c) a multivitamin formulation.

In certain aspects, the pharmaceutical composition is formulated repeated or continuous administration. In certain aspects, the pharmaceutical composition is formulated as a food fit for a mammal. The pharmaceutical composition may be formulated as a nutrient bar. In certain aspects, the A2AR pathway agonist is a specific A2AR agonist. In some embodiments, the A2AR agonist binds A2AR. In some embodiments, the A2AR agonist is a small molecule that binds A2AR.

Herein is also disclosed a packaged pharmaceutical preparation comprising: (a) a pharmaceutically acceptable carrier, (b) a therapeutically effective amount of an A2AR pathway agonist, and (c) a label stating that the pharmaceutical preparation is intended for the treatment of obesity. Herein Applicants also provide a packaged pharmaceutical preparation comprising: (a) a pharmaceutically acceptable carrier, (b) a therapeutically effective amount of an A2AR pathway agonist, and (c) a label stating that the pharmaceutical preparation is intended for preventing weight gain.

Applicants also provide, inter alia, packaged pharmaceutical preparation comprising: (a) a pharmaceutically acceptable carrier, (b) a therapeutically effective amount of an A2AR pathway agonist, and (c) a label stating that the pharmaceutical preparation is intended for inducing satiety. Also provided is a packaged pharmaceutical preparation comprising: (a) a pharmaceutically acceptable carrier, (b) a therapeutically effective amount of an A2AR pathway agonist, and (c) instructions and/or a label stating that the pharmaceuticals preparation is intended to be taken before sleeping.

In certain embodiments, the A2AR pathway agonist is administered conjointly with a therapy known to cause weight gain. This weight gain may be associated with the administration of a diabetes treatment, an antidepressant, a steroid or a hormone, a beta blocker, an alpha blocker, or a contraceptive.

The diabetes treatment may be at least one of the following: a sulfonylurea, a thiazolidinedione, a meglitinide, nateglinide, repaglinide, or insulin. The antidepression may be at least one of the following: a tricyclic antidepressant, an irreversible monoamine oxidase inhibitor (MAOI), a selective serotonin reuptake inhibitor (SSRI), bupropion, paroxetine, or mirtazapine.

In some aspects, the present disclosure provides compositions comprising at least one A2AR pathway agonist and at least one drug that induces weight gain. In certain embodiments, the drug that induces weight gain is at least one of the following: an anti-diabetic (e.g., a sulfonylurea, a thiazolidinedione, a meglitinide, nateglinide, repaglinide, or insulin), an antidepressant (e.g., a tricyclic antidepressant, an irreversible monoamine oxidase inhibitor (MAOI), a selective serotonin reuptake inhibitor (SSRI), bupropion, paroxetine, or mirtazapine), a steroid, a hormone, a beta blocker, an alpha blocker, or a contraceptive. In exemplary embodiments, the composition may comprise a therapeutically effective amount of at least one A2AR pathway agonist and a therapeutically effective amount of at least one drug that induces weight gain.

In some embodiments, the A2AR pathway agonist is a compound according to Formula (I) or a pharmaceutically acceptable salt thereof:

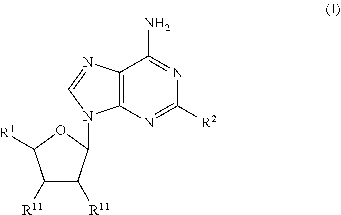

wherein:

R¹ is —C(O)NR³R⁴;

each R¹¹ is independently selected from —H, and OR⁵;

R⁵ is —H, C₁₋₄ alkyl, —C(O)C₁₋₄alkyl, or —C(O)H;

R² is selected from —H, and —NR⁶-C₁₋₄alkyl-phenyl-C₁₋₄alkyl wherein said alkyl groups are optionally substituted with —COOR⁷, or —CONR⁸R⁹;

R³, R⁴, R⁶, R⁷, R⁸, and R⁹ are each independently —H, —C₁₋₄alkyl, or -C₁₋₄alkyl-NH₂.

### BRIEF DESCRIPTION OF THE DRAWINGS

FIG. 1 is a chart depicting the weight of mice (y axis) versus age (x axis). Mice were either fed a low-fat or high-fat diet and were either treated or not treated with the selective A2AR agonist CGS21680. Data points marked with diamonds represent the weight of a mouse fed a low-fat diet and not treated with CGS21680. Data points marked with squares represent the weight of a mouse fed a low-fat diet and treated with CGS21680. Data points marked with triangles represent the weight of a mouse fed a high-fat diet and not treated with CGS21680. Data points marked with crosses represent the weight of a mouse fed a high-fat diet and treated with CGS21680.

FIG. 2 depicts epididymal fat in mice deficient for A2AR. The top row depicts dissected epididymal fat from five different wild-type mice. These are age-matched and genetically matched control mice that were kept in identical conditions. The bottom row depicts dissected epididymal fat from five different age-matched mice in which A2AR was knocked out. The increased fat in the A2AR-deficient mice indicates that A2AR signaling promotes leanness, and loss of A2AR signaling promotes obesity.

FIG. 3 shows the body weight of mice fed a high-fat or low-fat diet and treated or untreated with an A2AR agonist, CGS21680. Left panel, mice fed a high-fat diet. Right panel, mice fed a low fat diet.

FIG. 4 depicts leptin levels in mice treated with an A2AR agonist, compared to untreated control mice.

FIG. 5 depicts reduction or prevention of weight gain by various A2AR agonists in mice consuming a high-fat diet.

FIG. 6 depicts certain A2AR agonists.

### DETAILED DESCRIPTION

### I. Definitions

An alkyl group is a straight chained or branched hydrocarbon which is completely saturated. Typically, a straight chained or branched alkyl group has from 1 to about 20 carbon atoms, preferably from 1 to about 10. Examples of straight chained and branched alkyl groups include methyl, ethyl, n-propyl, iso-propyl, n-butyl, sec-butyl, tert-butyl, pentyl, hexyl, pentyl and octyl. A C₁-C₄ straight chained or branched alkyl group is also referred to as a “lower alkyl” group.

As used herein, “A2AR pathway agonist” refers to any agent that promotes signaling in the A2AR pathway. An A2AR pathway agonist may promote A2AR pathway signaling by, for example, increasing levels of extracellular adenosine, increasing the number of A2A adenosine receptors per cell, and/or enhancing signaling by the A2A receptor. An A2AR pathway agonist may act on A2AR, upstream of A2AR, or downstream of A2AR. In certain embodiments, the A2AR pathway agonist is a selective A2AR agonist that modulates A2AR signaling 2, 5, 10, 20, 50, 100, 200, 500, or 1000-fold more strongly than signaling of another pathway, such as histamine receptor signaling (such as H3 or H4 histamine receptors), adrenergic receptor signaling (such as β2, β3, or 132 adrenergic receptors), PDE4 signaling, cholinergic muscarinic receptor signaling, adenosine A1 receptor signaling, adenosine A2B receptor signaling, or adenosine A3 receptor signaling. In certain embodiments, the selective A2AR agonist has a K_{d} that is less than ½, ⅕, 1/10, 1/20, 1/50, 1/100, 1/200, 1/500, or 1/1000 the K_{d} of the agonist for another receptor, such as a histamine receptor (such as H3 or H4 histamine receptors), an adrenergic receptor (such as β2, β3, or 132 adrenergic receptors), PDE4, a cholinergic muscarinic receptor, an adenosine A1 receptor, an adenosine A2B receptor, or an adenosine A3 receptor. In certain embodiments, the A2AR pathway agonist is a specific agonist of A2AR. The A2AR pathway agonist may also be a small molecule that binds A2AR. This binding may be covalent or noncovalent. In certain embodiments, the A2AR pathway agonist binds to A2AR with a K_{d} of less than 1 μM, 500 nM, 200 nM, 100 nM, 50 nM, 20 nM, 10 nM, 5 nM, 2 nM, or 1 nM. In certain embodiments, the A2AR pathway agonist induces signal transduction pathways characteristic of adenosine binding to A2AR. For example, the A2AR pathway agonist may induce an increase in cAMP levels relative to a control cell or tissue under similar conditions that is not treated with an A2AR pathway agonist. In addition, said agonist may increase PKA activation and/or phosphorylation of CREB relative to a control cell or tissue.

The term “agent” is used herein to denote a chemical compound, a mixture of chemical compounds, a biological macromolecule (such as a nucleic acid, an antibody, a protein or portion thereof, e.g., a peptide, including nucleic acid mimetics and peptidomimetics), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The term “animal” includes both humans and non-human animals. The animal may be overweight or obese. The animal may be predisposed or susceptible to overweight or obesity.

As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can benefit from a combined effect of different therapeutic compounds. Similarly, an individual receiving “conjoint treatment” with two or more treatments is an individual that receives said treatments such that the second treatment is administered when the effects of the first treatment are still present in the body. Synergistic effects of the two treatments may be observed.

“Diet regimen”, as used herein, refers to a program that regulates the amount of food an individual consumes, in order to manage body weight. In some instances, the diet regimen involves a reduced number of calories per day compared to an individual's daily caloric intake prior to the diet regimen. A diet regimen may also include reduced fat, sugar, and/or carbohydrate intake.

“Exercise regimen”, as used herein, refers to a program that regulates the amount of exercise an individual performs, in order to manage body weight. In some instances, an exercise regimen involves cardiovascular exercise.

As used herein, an “individual” refers to an animal in need of treatment or administration with a composition described herein. In certain embodiments, the individual is non-obese. In certain embodiments, the individual is a human. Alternatively, the individual may be a non-human animal. Non-human animals include farm animals (e.g., cows, horses, pigs, sheep, goats) and companion animals (e.g., dogs, cats). An individual in need of treatment with an A2AR agonist may be an individual who is obese, likely to become obese, overweight, or likely to become overweight. Individuals who are likely to become obese or overweight can be identified, for example, based on family history, genetics, diet, activity level, medication intake, or various combinations thereof.

The term “induce drowsiness” refers to the decreasing of alertness or wakefulness. The term “induce drowsiness” includes promoting sleep.

The term “mimetic” as used herein refers to an agent having one or more of the same biological properties as the agent it mimics. For example, a leptin mimetic may be a polypeptide or oligopeptide having the same biological properties as leptin. In certain embodiments, the leptin mimetic binds the leptin receptor LepRb and activated LepRb as strongly as does leptin (within plus or minus 10, 20, or 50%). One readout of LepRb activation is that stat3 becomes phosphorylated.

As used herein, the term “promoting sleep” refers to increasing the quality or quantity of sleep. For example, promoting sleep can increase the ability to fall asleep or stay sleep, increase the number of hours slept prior to waking and increasing the perceived depth or refreshing effect of sleep. A compound that promotes sleep can, for example, cause the animal to sleep, prolong periods of sleep, promote restful sleep, decrease sleep latency, or decrease unwanted wake-like characteristics, such as anxiety and hyperactivity.

As used herein, the term “promoting wakefulness” refers to a causing a decrease in sleepiness, tendency to fall asleep, or other symptoms of undesired or reduced alertness or consciousness compared with sleepiness, tendency to fall asleep, or other symptoms of undesired or reduced alertness or consciousness expected or observed without treatment. Promoting wakefulness refers to a decrease in any stage of sleep, including light sleep, deeper sleep characterized by the presence of high amplitude, low wave brain activity termed “slow wave sleep”, and rapid eye movement (REM) sleep. A compound that promotes wakefulness can, for example, cause the animal to wake from sleep, prolong periods of wakefulness, prolong normal latency to sleep, restore normal sleep patterns following sleep deprivation, or enhance beneficial wake-like characteristics, such as alertness, responsiveness to stimuli, and energy.

As used herein, the term “small molecule” refers to an organic molecule with a relatively low molecular weight, e.g., less than about 1000 daltons. The term is used to differentiate these organic molecules from typical large biomolecules like nucleic acids, proteins, and complex carbohydrates like heparin and starch.

As used herein, “obesity” refers to a condition whereby a human has a Body Mass Index (BMI), which is calculated as weight (kg) per height (meters), of at least 25.9. Conventionally, those persons with normal weight (“non-obese individuals”) have a BMI of 19.9 to less than 25.9. One of skill in the art will be aware that definitions of obesity may very depending on the mammal in question. Methods of determining obesity in non-human animals are known in the art.

The phrase “pharmaceutically acceptable carrier” is art-recognized, and includes, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “preventing” and “limiting” are art-recognized, and when used in relation to a condition, such as obesity or any other medical condition, is well understood in the art, and includes administration of a composition which reduces the frequency of, or delays the onset of, symptoms of a medical condition in a subject relative to a subject which does not receive the composition. Thus, the “prevention” and “limiting” of obesity include, for example, reducing the weight gain and/or abdominal fat accumulation of non-obese patients receiving a prophylactic treatment relative to an untreated control population, and/or delaying weight gain and/or abdominal fat accumulation in a treated population versus an untreated control population, e.g., by a statistically and/or clinically significant amount.

When used with respect to a pharmaceutical composition or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, mucus secretions, lymph or the like, one or more of the pharmaceutically acceptable excipients may undergo gradual or delayed degradation (e.g., through hydrolysis) with concomitant release of any material incorporated therein, e.g., an therapeutic and/or biologically active salt and/or composition, for a sustained or extended period (as compared to the release from a bolus). This release may result in prolonged delivery of therapeutically effective amounts of any of the therapeutic agents disclosed herein.

The phrases “systemic administration,” “administered systemically,” “peripheral administration” and “administered peripherally” are art-recognized, and include the administration of a subject composition, therapeutic or other material at a site remote from the disease being treated. Administration of an agent directly into, onto, or in the vicinity of a lesion of the disease being treated, even if the agent is subsequently distributed systemically, may be termed “local” or “topical” or “regional” administration, other than directly into the central nervous system, e.g., by subcutaneous administration, such that it enters the patient's system and, thus, is subject to metabolism and other like processes.

The phrase “therapeutically effective amount” is an art-recognized term. In certain embodiments, the term refers to an amount of a salt or composition disclosed herein that produces some desired effect at a reasonable benefit/risk ratio applicable to any medical treatment. In certain embodiments, the term refers to that amount necessary or sufficient to eliminate or reduce medical symptoms for a period of time. The effective amount may vary depending on such factors as the disease or condition being treated, the particular targeted constructs being administered, the size of the subject, or the severity of the disease or condition. One of ordinary skill in the art may empirically determine the effective amount of a particular composition without necessitating undue experimentation. In some aspects, a therapeutically effective amount of an A2AR pathway agonist is the amount necessary to prevent weight gain in an individual.

The term “treating” is art-recognized and includes inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. The term “treating”, “treat” or “treatment” as used herein includes curative, adjunct and palliative treatment.

Preventative or prophylactic treatment means preventing a disease, disorder or condition from occurring in an animal which may be predisposed to the disease, disorder and/or condition but has not yet been diagnosed as having it;

As used herein, the term “weight gain” refers to an increase in body weight by at least 5 pounds. In some embodiments, the term “weight gain” refers to an increase in body weight by at least 10, 15, 20, 25, 30, 35, 40, 45, or 50 pounds.

### Exemplary A2AR Pathway Agonists

A2AR pathway agonists fall into a few basic categories. They can be adenosine mimetics, agents that prevent the breakdown or degradation of adenosine, adenosine deaminase inhibitors, adenosine kinase inhibitors, agonists of a Gs protein coupled receptor, cAMP mimetics, inhibitors of cAMP inactivation, agonists of adenylate cyclase, and/or A2AR agonists. These categories are not mutually exclusive.

In certain embodiments, the A2AR pathway agonist is adenosine, an adenosine prodrug, or an adenosine mimetic. Adenosine mimetics include N-ethylcarboxamidoadenosine (NECA) (U.S. Pat. No. 5,500,428), Polyadenylic acid (Todd J et al., “Intravascular Adenosine at Reperfusion Reduces Infarct Size and Neutrophil Adherence” Ann Thorac Surg 1996;62:1364-1372), 2-chloroadenosine (Camosa B G A et al, “The potentiation of the histamine release induced by adenosine in mast cells from guinea pig lung and heart: sharp dependence on the time of preincubation”, Pharmacological Research, Volume 41, Number 3, March 2000, pp. 291-297(7)). In some embodiments, the A2AR agonist is N⁶-(4-Amino-3 -iodobenzyl)adenosine-5 -N-ethylcarboxamidoadenosine bistrifluoroacetic acid. In some embodiments, the A2AR pathway agonist is not adenosine. In some embodiments, the A2AR pathway agonist is at least 2, 5, or 10, 20, or 50-fold more stable then adenosine.

In some embodiments, the A2AR pathway agonist stimulates adenosine synthesis. For example, it may be an activator of an enzyme that converts IMP into AMP (such as adenylosuccinate synthase and adenylosuccinate lyase). It may also be an activator of an enzyme that converts AMP to adenosine, such as 5′-nucleotidase. 5′-nucleotidase is activated by elevated PKC levels, so a PKC activator may be used to increase adenosine levels. Also, certain ions such as Mn²⁺ and zinc activate 5′-nucleotidase. The A2AR pathway agonist may also be an agent that increases the levels of an enzyme involved in adenosine synthesis.

In yet other embodiments, the A2AR pathway agonist prevents the breakdown or degradation of adenosine. Such agents include adenosine kinase inhibitors, adenosine deaminase inhibitors, and adenosine aminohydrolase inhibitors. Examples of adenosine kinase inhibitors arc well known in the art and include 5′-amino-5′-deoxyadenosine, 5-iodotubercidin, and 5′-deoxy-5-iodotubercidin, 4-(N-phenylamino)-5-phenyl-7-(5′-deoxyribofuranosyl)pyrrolo[2,3-d]pyrimidine (Wiesner J B et al., “Adenosine Kinase Inhibitors as a Novel Approach to Anticonvulsant Therapy”, Pharmacology, Vol. 289, Issue 3, 1669-1677, June 1999); GP3966 (Boyer S et al., “Adenosine Kinase Inhibitors. 5. Synthesis, Enzyme Inhibition, and Analgesic Activity of Diaryl-erythro-furanosyltubercidin Analogues”, J. Med. Chem., 48 (20), 6430-6441, 2005. 10.1021/jm0503650 50022-2623(05)00365-1); P¹,P⁵-Di(Adenosine-5′)Pentaphosphate(Ap₅A) (Kurebayashi N et al., “P¹,P⁵-Di(Adenosine-5)Pentaphosphate(Ap₅A) as an Inhibitor of Adenylate Kinase in Studies of Fragmented Sarcoplasmic Reticulum from Bullfrog Skeletal Muscle” J. Biochem, 1980, Vol. 88, No. 3 871-876); certain pyridopyrimidine analogues (Zheng G Z et al., “Pyridopyrimidine analogues as novel adenosine kinase inhibitors” Bioorganic & Medicinal Chemistry Letters, Volume 11, Issue 16, 20 Aug. 2001, Pages 2071-2074); GP-515 (Bulut K et al., “Long-Term Effects of the Adenosine Kinase Inhibitor GP-515 on Hepatic Microcirculation Following Hemorrhagic Shock” European Journal of Trauma, Volume 29, Number 3, June, 2003). Additional adenosine kinase inhibitors are disclosed in U.S. Pat. No. 5,721,356. Adenosine kinase inhibitors also include nucleic acids (such as siRNAs) designed to downregulate adenosine kinase.

Adenosine deaminase inhibitors include nucleic acids (such as siRNAs) designed to downregulate adenosine deaminase. Additional adenosine deaminase inhibitors include erythro-9-(2-hydroxy-3-nonyl)adenine (EHNA) and coformycin (Sandhu G S et al., “Adenosine deaminase inhibitors attenuate ischemic injury and preserve energy balance in isolated guinea pig heart”, Am J Physiol Heart Circ Physiol 265: H1249-H1256, 1993); 2′-Deoxycoformycin (Schrier S M et al., Biochem Pharmacol. 2001 Feb. 15; 61(4):417-25). Adenosine deaminase inhibitors are also disclosed in U.S. Pat. No. 5,731,432.

In certain aspects, the A2AR pathway agonist is an agonist of a Gs protein coupled receptor. For example, it may be a small molecule that binds to and activates the Gs protein coupled receptor. The A2AR pathway agonist may also be a nucleic acid that encodes the Gs protein coupled receptor.

In some embodiments utilizing an A2AR pathway agonist, it can be a cAMP mimetic. Exemplary cAMP mimetics include PKA activators and adenylate cyclase activators. In other embodiments, the A2AR pathway agonist is an inhibitor of cAMP-degradation such as a cAMP phosphodiesterase inhibitor. Exemplary cAMP phosphodiesterase inhibitors include theophylline, denbutyline, XT-44, roflumilast, revizinone, pimobendan, olprinone, cilomilast, piclamilast, hydroxynonyladenine, motapizone, and dipyridamole (PCT application WO02069905A2) the compounds disclosed in US Patent Application No. US20070117861A1, adenosine-3′,5′-cyclic monophosphorothioate Sp-isomer (Sp-cAMP) (Sheriff S et al., “Hypothalamic administration of cAMP agonist/PKA activator inhibits both schedule feeding and NPY-induced feeding in rats”, Peptides, Volume 24, Number 2, February 2003, pp. 245-254(10)); (Bu)₂cAMP, 8-br-cAMP, epinephrine, pituitary adenylate cyclase-activating polypeptide (PACAP) (Bousquet C et al., “cAMP Neuropeptide Agonists Induce Pituitary Suppressor of Cytokine Signaling-3: Novel Negative Feedback Mechanism for Corticotroph Cytokine Action”, Molecular Endocrinology 15 (11): 1880-1890); and dbcAMP (Huang Y H et al., “Signals of seminal vesicle autoantigen suppresses bovine serum albumin-induced capacitation in mouse sperm” Biochemical and Biophysical Research Communications Volume 338, Issue 3, 23 Dec. 2005, Pages 1564-1571).

In certain embodiments, the A2AR pathway agonist stimulates adenylate cyclase activity. Such A2AR pathway agonists include forskolin and forskolin analogues (described in Laurenza A et al., “Stimulation of adenylate cyclase by water-soluble analogues of forskolin” Molecular Pharmacology Volume 32, Issue 1, pp. 133-139, Jul. 1, 1987). Other such agonists include guanosine 5′[βγ-imido]triphosphate, p[NH]ppG, fluoride. Yet other such agonists include oxymetazoline, UK-14304, BHT-933, BHT-920 (Eason M G et al., “Contribution of ligand structure to activation of alpha 2-adrenergic receptor subtype coupling to Gs”, Volume 45, Issue 4, pp. 696-702, Apr. 1, 1994).

In certain embodiments, the A2AR pathway agonist is an A2AR agonist. In certain embodiments, the A2AR pathway agonist is a small molecule that binds A2AR. This binding may be covalent or noncovalent. In certain embodiments, the A2AR pathway agonist is a selective agonist of A2AR. For instance, a selective A2AR pathway agonist may activate A2AR 2-fold, 5-fold, 10-fold, 20-fold, 50-fold, 100-fold, 200-fold, 500-fold, or 1000-fold or more strongly than it activates an A1 or A3 adenosine receptor.

In certain embodiments, the A2AR agonist is a compound according to Formula (I) or a pharmaceutically acceptable salt thereof:

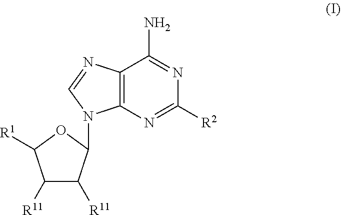

wherein:

R¹ is —C(O)NR³R⁴;

each R¹¹ is independently selected from —H, and OR⁵;

R⁵ is —H, C₁₋₄ alkyl, —C(O)C₁₋₄alkyl, or —C(O)H;

R² is selected from —H, and —NR⁶-C₁₋₄alkyl-phenyl-C₁₋₄alkyl wherein said alkyl groups are optionally substituted with —C(O)OR⁷, or —C(O)NR⁸R⁹; and

R³, R⁴, R⁶, R⁷, R⁸, and R⁹ are each independently —H, —C₁₋₄alkyl, or —C₁₋₄alkyl-NH₂.

In certain embodiments, R¹ is —CONHC₁₋₄alkyl, where the alkyl group is methyl, ethyl, or propyl. In another embodiment, R¹ is —CONHCH₂CH₃.

In certain embodiments, R¹¹ is —H, —OCH₃, —OC(O)CH₃, or —OH. In another embodiment, each R¹¹ is —OH.

In certain embodiments R² is —H or —NHCH₂CH₂-phenyl-CH₂CH₃, where the alkyl groups are optionally substituted by —C(O)OH or C(O)NHC₁₋₄alkyl-NH. In certain embodiments, R² is —H, —NHCH₂CH₂-phenyl-CH₂CH₂—C(O)OH, or —NHCH₂CH₂-phenyl-CH₂CH₂—C(O)NH—CH₂—CH₂—NH₂.

In some embodiments, a compound of Formula (I) is 3-[4-[2-[[6-amino-9-[(2R,3R,4S,5S)-5-(ethylcarbamoyl)-3,4-dihydroxy-oxolan-2-yl]purin-2-yl]amino]ethyl]phenyl]propanoic acid (CGS21680). In some embodiments, a compound of Formula (I) is 2-[(2- aminoethylamino)carbonylethylphenylethylamino]-5′-N-ethylcarboxamidoadenosine (APEC). In some embodiments, a compound of Formula (I) is 5′-N-ethylcarboxamidoadenosine or 1-(6-Amino-9H-purin-9-yl)-1-deoxy-N-ethyl-β-D-ribofuranuronamide (NECA).

Certain adenosine A2A receptor agonists useful in the methods herein may be selected from the group consisting of 2-phenylaminoadenosine, 2-para-2-carboxyethylphenylamino-5′-N- ethylcarboxamido-adenosine, 5′-N-ethylcarboxamidoadenosine, 5′-N-cyclopropyladenosine, 5′-N- methylcarboxamidoadenosine and PD-125944 (for chemical structures, see Bruns, R. F., Ann. N.Y. Acad. Sci. 603:211-226 (1990) at page 216).

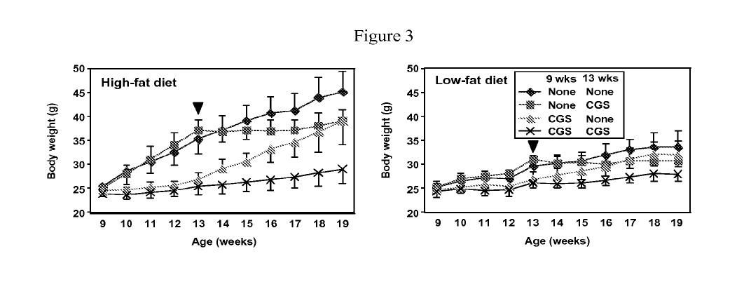

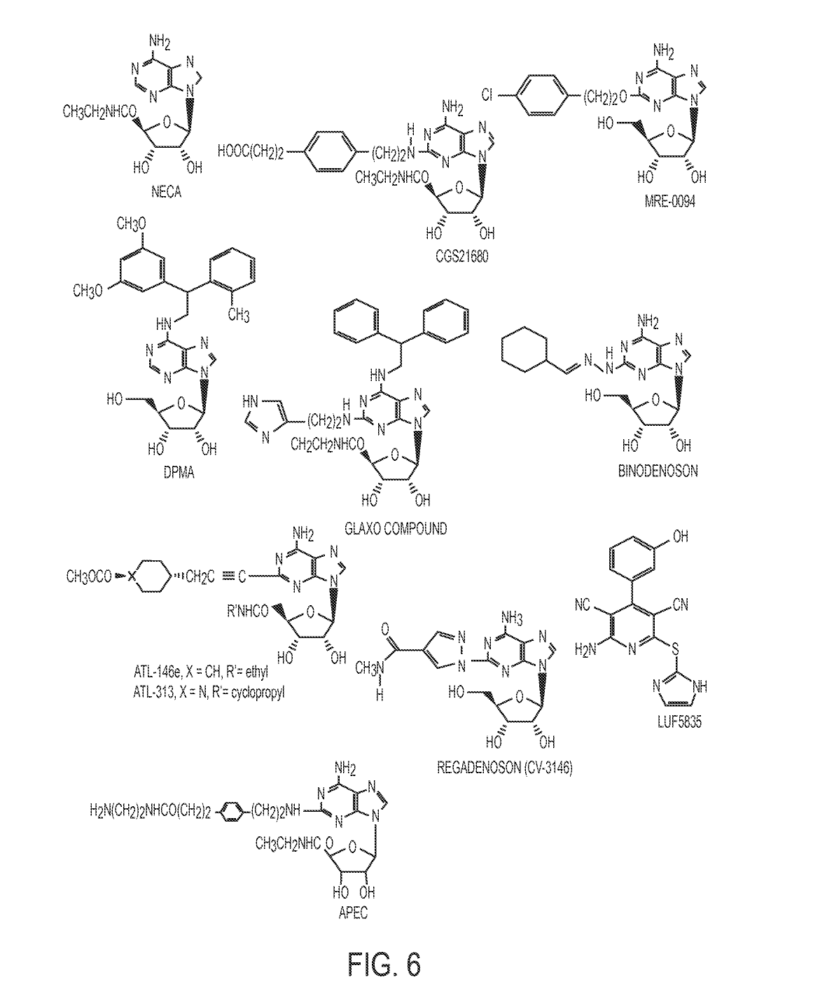

Exemplary A2AR agonists include NECA, CGS21680, MRE-0094, DPMA, Glaxo compound (structure provided in FIG. 3), Binodenoson (MRE-0470), ATL-146e (4-[3-[6-amino-9-(5-ethylcarbamoyl-3,4-dihydroxy-tetrahydro-furan-2-yl)-9H-purin-2-yl]-prop-2-ynyl]-cyclohexanecarboxylicacid methyl ester), regadenoson (CVT3146), ATL-313, GW328267X, CV-3146, CVT-3033, LUF5835, Apadenoson, CGS 22492C, and MRA470. The structures of certain of these A2AR agonists are illustrated in FIG. 6. Other adenosine pathway drugs that may be used in accordance with the methods herein include those produced by Adenosine Therapeutics LLC (ATL1222, ATL844, ATL9844, ATL908, ATL902, ATL202, and ATL359). Additional A2AR agonists include 2-[(2-aminoethylamino)carbonylethylphenylethylamino]-5′-N- ethylcarboxamidoadenosine (APEC), N6-cyclohexyladenosine (CHA) (Nikodijevic O, “Behavioral effects of A1- and A2-selective adenosine agonists and antagonists: evidence for synergism and antagonism”, Journal of Pharmacology, and Experimental Therapeutics, Volume 259, Issue 1, pp. 286-294, Oct. 1, 1991), and 6-[(2,2-diphenylethyl)amino]-9-(N-ethyl-beta-D-ribofuranosyluronamide)-N-(2-{N′-[1-(2-pyridyl)-4-piperidyl]ureido}ethyl)-9H-purine-2-carboxamide (European Patent No. EP1456219A1; Pfizer), 2-[(cyclohexylmethylene)hydrazino]adenosine (MRE-0470) (Martin P L et al., “Pharmacology and toxicology of the A2A-adenosine receptor agonist 2-[(cyclohexylmethylene)hydrazino]adenosine (MRE-0470) in the rat” Drug Development Research, Volume 42, Issue 2, Pages 76 -85). Additional A2AR agonists are described in the following publications: U.S. Pat. No. 6,495,528, WO9967266A1, WO05116037A1, WO07009757A1, WO07059949A1, WO8803147A1, U.S. Pat. No. 4,657,897, U.S. Pat. No. 4,657,898, U.S. Pat. No. 4755594, U.S. Pat. No. 4,714,697, U.S. Pat. No. 4,673,670, U.S. Pat. No. 4,614,732, U.S. Pat. No. 4,764,506, U.S. Pat. No. 4,683,223, U.S. Pat. No. 4,636,493, U.S. Pat. No. 4,600,707, U.S. Pat. No. 4,791,103, U.S. Pat. No. 4,780,464, U.S. Pat. No. 7,238,676, U.S. Pat. No. 6,921,753, US20050124574A1, U.S. Pat. No. 6,900,309, US20040229838A1, US20040229838A1, U.S. Pat. No. 6,624,158, U.S. Pat. No. 6,525,032, U.S. Pat. No. 6,448,236, WO0222630A1, U.S. Pat. No. 6,350,735, WO0200676A1, WO0160835C1, WO0160835A1, WO0127131A1, WO0127130A1, WO0077018A3, WO0077018A2, U.S. Pat. No. 4,738,954, U.S. Pat. No. 4,501,735, U.S. Pat. No. 4,663,313, U.S. Pat. No. 4,616,003, WO8803148A3, U.S. Pat. No. 4,837,207, WO03048180A1, WO02096462A1, U.S. Pat. No. 6,326,359, WO0023457A1, WO06023272A1, WO06028618A1, WO03029264A3, WO05107463A1, WO9934804A1, WO0078774A3, WO0078774A2, WO0072799A3, U.S. Pat. No. 5,877,180, U.S. Pat. No. 6,448,235, US20080027022A1, WO07120972A3, US20070265440A1, U.S. Pat. No. 7,226,913, WO03086408A1, US20080064653A1, U.S. Pat. No. 5,075,290, U.S. Pat. No. 7,183,264, U.S. Pat. No. 7,144,872, U.S. Pat. No. 7,109,180, U.S. Pat. No. 6,770,634, U.S. Pat. No. 6,642,210, U.S. Pat. No. 6,440,948, U.S. Pat. No. 6,403,567, U.S. Pat. No. 6,214,807, U.S. Pat. No. 6,180,615, US20070207978, US20070203090, US20060052332, US20040198692, US20040038928, and US20040038928.

A number of other A2A receptor agonists have been described, such as substituted 4′-carboxamido and 4′-thioamido adenosine derivatives, in International Patent Application Nos. WO94/17090, WO96/02553, and WO96/02543. Also, certain selective A2AR agonists are described in International Patent Application Nos. WO98/28319, WO99/38877, WO99/41267, WO99/67263, WO99/67264, WO99/67265 and WO99/67266, WO00/23457, WO00/77018, WO01/94368 and WO02/00676. A2A receptor agonists have also been described in WO00/78776, WO00/78777, WO00/78778, WO00/78779, WO00/72799 and U.S. Pat. No. 5,877,180.

In certain embodiments, the A2AR agonist is: N-({9-[(2R,3R,4S,5R)-3,4-dihydroxy-5-(methoxymethyl)tetrahydro-2-furanyl-6[(2,2- diphenylethyl)amino]-9H-purin-2-yl}methyl)-2-methyl-1-propanesulfonamide (Example 15 of WO00/23457); cis-(2R,3R,4S,5R)-2-(6-[(2,2-diphenylethyl)amino]-2-{[(4-isopropylcyclohexyl)amino]methyl}-9H-purin-9-yl)-5-(methoxymethyl)tetrahydro-3,4-furandiol and trans-(2R,3R,4S,5R)-2-(6-[(2,2-diphenylethyl)amino]-2-{[(4-isopropylcyclohexyl)amino]methyl}-9H-purin-9-yl)-5-(methoxymethyl)tetrahydro-3,4-furandiol (Example 17 of WO00/23457); N-({9-[(2R,3R,4S,5R)-3,4-dihydroxy-5-(hydroxymethyl)tetrahydro-2-furanyl]-6[(2,2-diphenylethyl)amino]-9H-purin-2-yl}methyl)-2-methyl-1-propanesulfonamide (Example 1 of WO01/27130); (2S,3S,4R,5R)-5-(6-[(2,2-diphenylethyl)amino]-2-{[(isopropylsulfonyl)amino]methyl}-9H-purin-9-yl)-N-ethyl-3,4-dihydroxytetrahydro-2-furancarboxamide (Example 3 of WO01/27131); 9-[(2R,3R,4S,5R)-3,4-dihydroxy-5-(hydroxymethyl)tetrahydro-2-furanyl]-6-[(2,2 diphenylethyl)amino]-N-[2-(1-piperidinyl)ethyl]-9H-purine-2-carboxamide (Example 1 of WO00/77018); 6-[(2,2-diphenylethyl)amino]-9-{(2R,3R,4S,5S)-5-[(ethylamino)carbonyl]-3,4-dihydroxytetrahydro-2-furanyl}-N-[2-(1-piperidinyl)ethyl]-9H-purine-2 carboxamide (Example 1 of WO01/60835); N-({9-[(2R,3R,4S,5R)-3,4-dihydroxy-5-(hydroxymethyl)tetrahydro-2-furanyl-6-[(2,2-diphenylethyl)amino]-9H-purin-2-yl}methyl)-N′-2-(diisopropylamino)ethyl]urea (from Example 1 of WO02/00676); or 6-[(2,2-diphenylethyl)amino]-9-{(2R,3R,4S,5S)-5-[(ethylamino)carbonyl]-3,4-dihydroxytetrahydro-2-furanyl}-N-{2-[({[1-(2-pyridinyl)-4-piperidinyl]amino}carbonyl)amino]ethyl}-9H-purine-2-carboxamide or a pharmaceutically acceptable salt or solvate thereof.

A number of A2AR agonists, and methods of making them, are described in U.S. Patent Application No. US20070270373A1, such as a compound of Formula (II) below:

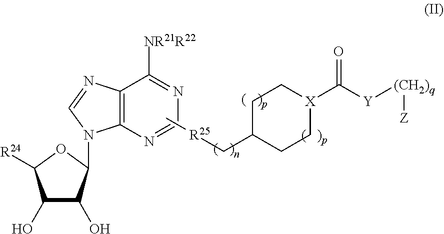

wherein:

R²¹ and R²² independently are selected from the group consisting of H, (C₁-C₈)alkyl, (C₃-C₈)cycloalkyl, (C₃-C₈)cycloalkyl(C₁-C₈)alkylene, aryl, aryl(C₁-C₈)alkylene, heteroaryl, heteroaryl(C₁-C₈)alkylene-, diaryl(C₁-C₈)alkylene, and diheteroaryl(C₁-C₈)alkylene, wherein the aryl and heteroaryl rings are optionally substituted with 1-4 groups independently selected from fluoro, chloro, iodo, bromo, methyl, trifluoromethyl, and methoxy;

each R independently is selected from the group consisting of H, C₁-C₄alkyl, cyclopropyl, cyclobutyl, and (CH₂)ₐcyclopropyl;

X is CH or N, provided that when X is CH then Z cannot be substituted with halogen, C₁-C₆ alkyl, hydroxyl, amino, or mono- or di-(C₁-C₆-alkyl)amino;

Y is selected from the group consisting of O, NR²¹, OCH₂CH₂O)ₘCH₂—, and —(NR¹CH₂CH₂O)ₘCH₂—, provided that when Y is O or NR²¹, then at least one substituent is present on Z;

Z is selected from the group consisting of 5-membered heteroaryl, 6-membered aryl, 6-membered heteroaryl, carbocyclic biaryl, and heterocyclic biaryl, wherein the point of attachment of Y to Z is a carbon atom on Z, wherein Z is substituted with 0-4 groups independently selected from the group consisting of F, Cl, Br, I, (C₁-C₄)alkyl, —(CH₂)ₐOR²³, —(CH₂)ₐNR²³R²³, —NHOH, —NR²³NR²³R²³, nitro, —(CH₂)ₐCN, —(CH₂)ₐCO₂R²³, —(CH₂)ₐCONR²³R²³, trifluoromethyl, and trifluoromethoxy;

alternatively, Y and Z together form an indolyl, indolinyl, isoindolinyl, tetrahydroisoquinolinyl, or tetrahydroquinolinyl moiety wherein the point of attachment is via the ring nitrogen and wherein said indolyl, indolinyl, isoindolinyl, tetrahydroisoquinolinyl, or tetrahydroquinolinyl moiety, which is substituted with 0-4 groups independently selected from the group consisting of F, Cl, Br, I, C₁-C₄alkyl, —(CH₂)ₐOR²³, —(CH₂)ₐNR²³R²³, —NHOH, —NR²³NR²³R²³, NO₂, —(CH₂)ₐCN, —(CH₂)ₐCO₂R²³, —(CH₂)ₐCONR²³R²³, CF₃, and OCF₃;

R²³ is independently selected from the group consisting of H, (C₁-C₆)alkyl, cycloalkyl, aryl, and heteroaryl;

R²⁴ is selected from the group consisting of CH₂OR, C(O)NRR, and CO₂R;

R²⁵ is selected from the group consisting of CH₂CH₂, CH═CH, and C≡C;

a is selected from 0, 1, and 2;

m is selected from 1, 2, and 3;

n is selected from 0, 1, and 2;

each p independently is selected from 0, 1, and 2; and,

q is selected from 0, 1, and 2.

In certain embodiments, the A2AR agonist that is a compound of Formula II above is a compound of Formula (IIa):

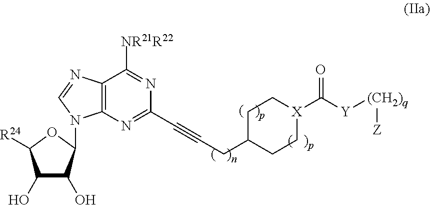

In certain embodiments, the A2AR agonist that is a compound of Formula IIa above is a compound of Formula IIb:

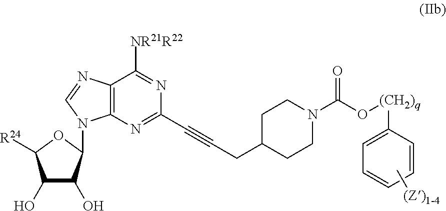

wherein:

each Z′ is independently selected from the group consisting F, Cl, Br, I, C₁-C₄alkyl, —(CH₂)ₐOR²³, —(CH₂)ₐNR²³R²³, —NHOH, —NW³NR²³R²³, NO₂, —(CH₂)ₐCN, —(CH₂)ₐCO₂R²³, —(CH₂)ₐCONR²³R²³, CF₃, and OCF₃.

In certain embodiments, the A2AR agonist that is a compound of Formula (IIb) above is a compound of Formula (IIc):

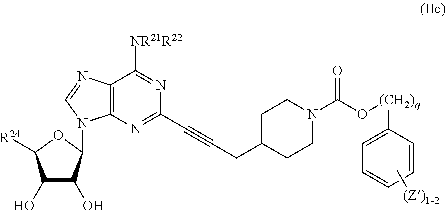

In certain embodiments, Z′ is selected from the group consisting of F, Cl, methyl, OR²³, NO₂, CN, NR²³R²³ and CO₂R²³. In some embodiments, R²³ is methyl or hydrogen.

In other embodiments, the A2AR agonist is an agonist described in US Patent Application No. 20070183995, such as that of Formula (III):

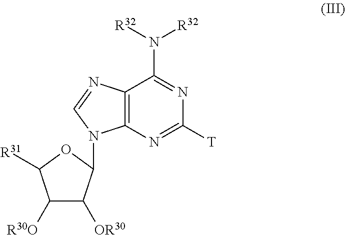

wherein:
   (a) each R³⁰ is independently hydrogen, a C₁-C₂₀ linear, branched, substituted, unsubstituted, saturated and/or unsaturated alkyl, acyl group or aryl group;
   (b) R³¹ is a C₁-C₅ alkanol or

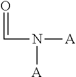

where each A is independently hydrogen or a C₁-C₅alkyl; and
   (c) T is a group comprising at least one heteroatom with the provisos that T has a heteroatom selected from the group consisting of N, O and S bonded to purine and when T is

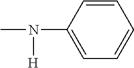

each R³⁰ and R³² are not simultaneously H when R³¹ is CH₂OH, and when T is

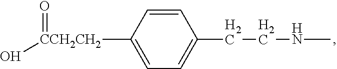

each R³⁰ and R³² are not simultaneously hydrogen when R³¹ is

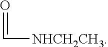

In certain embodiments, T is

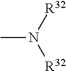

where each R³² is independently
(a) hydrogen, a C₁-C₂₀ linear, branched, cyclic, saturated or unsaturated alkyl group with or without a heteroatom selected from the group consisting of N, O and S, an aryl group, alkyl aryl, C₄-C₉ heteroaryl, C₄-C₁₀ heterocycle where the heteroatom is selected from the group consisting of N, O and S,

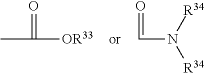

where R³³ is a C₁-C₂₀ linear, branched, saturated or unsaturated alkyl group with or without a heteroatom selected from the group consisting of N, O and S, and each R³⁴ is independently hydrogen, C₁-C₂₀ linear, branched, saturated or unsaturated alkyl group with or without a heteroatom selected from the group consisting of N, O and S, with the provisos that when T is

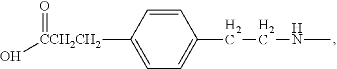

each R³⁰ and R³² are not simultaneously hydrogen when R³¹ is

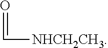

In some embodiments, the A2AR agonist is an agonist described in U.S. Pat. No. 6,642,210 (CV Therapeutics), such as compounds of Formula (IV), or a pharmaceutically acceptable salt thereof:

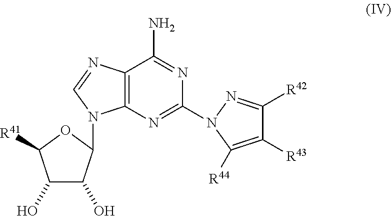

wherein R⁴¹═CH₂OH;
R⁴³ is selected from the group consisting of CO₂R⁴⁹, —CONR⁴⁷R⁴⁸, and aryl, wherein the aryl substituent is optionally substituted with from 1 to 3 substituents independently selected from the group consisting of halo, alkyl, and OR⁴⁹;
R⁴⁷ is selected from the group consisting of hydrogen, straight or branched C₁₋₁₅alkyl and C₃₋₈cycloalkyl, wherein the alkyl substituent is optionally substituted with from 1 to 3 substituents independently selected from the group consisting of aryl and CO₂R²⁰, and wherein the optional aryl substituent is optionally substituted with halo;
R⁴⁸ is selected from the group consisting of hydrogen, straight or branched C₁₁₅alkyl and C₃₋₈cycloalkyl;
R⁴⁹ is selected from the group consisting of hydrogen and C₁₋₁₅alkyl;
and wherein R⁴² and R⁴⁴ are hydrogen.

In certain embodiments relating to Formula IV, R³ is CO₂R²⁰; and R²⁰ is selected from the group consisting of hydrogen and C₁₋₄alkyl.

In some embodiments relating to Formula IV, R⁴³ is CONR⁴⁷R⁴⁸;
R⁴⁷ is selected from the group consisting of hydrogen, straight or branched C¹⁻¹⁰alkyl and C₃₋₅cycloalkyl, wherein the alkyl substituent is optionally substituted with from 1 to 2 substituents independently selected from the group consisting of aryl and CO₂R²⁰;
R⁴⁸ is selected from the group consisting of hydrogen, straight and branched C₁₋₃alkyl and C₃₋₅cycloalkyl; and
R⁴⁹ is C₁₋₄alkyl.

In certain embodiments relating to Formula (IV), R⁴³ is aryl, wherein the aryl substituent is optionally substituted with from 1 to 3 substituents independently selected from the group consisting of halo, alkyl and OR⁴⁹; and R⁴⁹ is selected from and the group consisting of C₁₋₄alkyl. Optionally, the compound described in the preceding sentence may be produced such that R⁴³ is aryl, wherein the aryl substituent is phenyl optionally substituted with from 1 to 2 substituents independently selected from the group consisting of chloro, methyl and OR⁴⁹; and R⁴⁹ is methyl.

In some embodiments relating to Formula (IV), R⁴³ is CO₂R⁴⁹; and R⁴⁹ is selected from the group consisting of hydrogen and C¹⁻⁴alkyl.

In certain aspects, R⁴⁷ is selected from the group consisting of hydrogen, C₁₋₃alkyl and cyclopentyl, wherein the alkyl substituent is optionally substituted with from 1 to 2 substituents, independently selected from the group consisting of phenyl and CO₂ R⁴⁹ and wherein each optional phenyl substituent is optionally substituted with halo; R⁴⁸ is selected from hydrogen and methyl; and R⁴⁹ is selected from hydrogen and ethyl.

In certain embodiments, the compound of Formula (IV) is selected from the group consisting of ethyl 1-{9-[(4S,2R,3R,5R)-3,4-dihydroxy-5-(hydroxymethyl)oxolan-2-yl]-6-aminopurin-2-yl}pyrazole-4-carboxylate; (4S,2R,3R,5R)-2-{6-amino-2-[4-(4-chlorophenyl)-pyrazolyl]purin-9-yl}-5-(hydroxymethyl)oxolane-3,4-diol; (4S,2R,3R,5R)-2-{6-amino-2-[4-(4-methoxyphenyl)pyrazolyl]purin-9-yl}-5-(hydroxymethyl)oxolane-3,4-diol; (4S,2R,3R,5R)-2-{6-amino-2-[4-(4-methylphenyl)pyrazolyl]purin-9-yl}-5-(hydroxymethyl)-oxolane-3,4-diol; (1-{9-[(4S,2R,3R,5R)-3,4-dihydroxy-5-(hydroxymethyl)oxolan-2-yl]-6-aminopurin-2-yl}pyrazol-4-yl)-N-methylcarboxamide; 1-{9-[(4S,2R,3R,5R)-3,4-dihydroxy-5-(hydroxyrethyl)oxolan-2-yl]-6-aminopurin-2-yl}pyrazole-4-carboxylic acid; (1-{9-[(4S,2R,3R,5R)-3,4-dihydroxy-5-(hydroxymethyl)oxolan-2-yl]-6-aminopurin-2-yl}pyrazol-4-yl)-N,N-dimethylcarboxamide; (1-{9-[(4S,2R,3R,5R)-3,4-dihydroxy-5-(hydroxymethyl)oxolan-2-yl]-6-aminopurin-2-yl}pyrazol-4-yl)-N-ethylcarboxamide; 1-{9-[(4S,2R,3R,5R)-3,4-dihydroxy-5-(hydroxymethyl)oxolan-2-yl]-6-aminopurin-2-yl}pyrazole-4-carboxamide; 1-{9-[(4S,2R,3R,5R)-3,4-dihydroxy-5-(hydroxymethyl)oxolan-2-yl]-6-aminopurin-2-yl}pyrazol-⁴-yl)-N-(cyclopentyl)carboxamide; (1-{9-[(4S,2R,3R,5R)-3,4-dihydroxy-5-(hydroxymethyl)oxolan-2-yl]-6-aminopurin-2-yl}pyrazol-4-yl)-N-[(4-chlorophenyl)methyl]carboxamide, and ethyl 2-[(1-{9-[(4S,2R,3R,5R)-3,4-dihydroxy-5-(hydroxymethyl)oxolan-2-yl]-6-aminopurin-2-yl}pyrazol-4-yl)carbonylamino]acetate. In certain embodiments, the A2AR agonist is Regadonoson.

In other embodiments, the A2AR agonist is a compound of Formula (V) or a pharmaceutically acceptable salt thereof, as described in US2006/0135466:

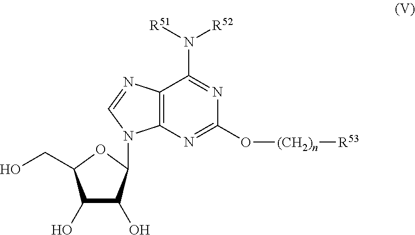

wherein:
R⁵¹ and R⁵² are independently selected from hydrogen, C₁-C₆alkyl, C₂-C₆alkenyl, C₂-C₆alkynyl, C₃-C₈cycloalkyl, C₃-C₈cycloalkenyl, C₇-C₁₂aralkyl, C₆-C₁₂aryl, 5-7 membered heteroaryl, 4-7 membered heterocycloalkyl, each of which is optionally substituted with 1 to 3 substituents independently selected from the group consisting of hydroxy, C₁-C₆alkyl, C₁-C₆alkoxy, amino, mono-(C₁-C₆alkyl)amino, di-(C₁-C₆alkyl)amino, halogen, hydroxy, cyano, nitro, carboxylate, carboxamide, sulfonate, and sulfonamide; or
NR⁵¹R⁵² taken in combination forms a 4-7 membered heterocycloalkyl or a 5-7 membered heteroaryl group, each of which is optionally substituted with 1 to 3 substituents independently selected from the group consisting of hydroxy, C₁-C₆alkyl, C₁-C₆alkoxy, amino, mono-(C₁-C₆alkyl)amino, di-(C₁-C₆alkyl)amino, halogen, hydroxy, cyano, nitro, carboxylate, carboxamide, sulfonate, and sulfonamide;
R⁵³ is aryl, cycloalkyl or heteroaryl, each of which is optionally substituted with 1 to 3 substituents independently selected from the group consisting of hydroxy, C₁-C₆alkyl, C₁-C₆alkoxy, amino, mono-(C₁-C₆alkyl)amino, di-(C₁-C₆alkyl)amino, halogen, hydroxy, cyano, nitro, carboxylate, carboxamide, sulfonate, and sulfonamide; and
n is an integer of 2 or 3.

In certain embodiments, the A2AR agonist is MRE-0094.

In certain embodiments, the A2AR pathway agonist is an adenosine kinase inhibitor such as those described in PCT Publication No. WO9640707A1. For instance, the adenosine kinase inhibitor may be of the general formula (VI), or a pharmaceutically acceptable salt thereof:

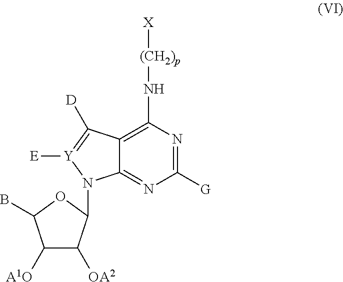

wherein:
A¹ and A² are each hydrogen or acyl, or together form a cyclic carbonate;
B is CH₃, alkenyl, or (CH₂)ₙ—B′, where n is from 1 to 4 and B′ is hydrogen, hydroxy, alkyl, alkoxy, amino, azido, halogen, or alkenyl;
D is halogen, alkyl, alkenyl, alkynyl, haloalkyl, cyano, carboxamido, or (CH₂)_{q}X where q is from 0 to 3;
and each X is independently an aryl group, more preferably an aromatic ring optionally containing a nitrogen, sulfur, or oxygen atom optionally substituted at any position by halogen, alkyl, alkoxy, substituted per halo lower alkyl, sulfonamide, cyano, CONRR′ where R and R′ are independently hydrogen or lower alkyl, or is a water solubilizing group (CH₂)ᵣT where r is from 0 to 3 and T is an alkyl or alkenyl chain of 0 to 16 carbon atoms containing a carboxylic acid and optionally containing one or more nitrogen atoms and optionally one or more oxygen atoms, a 5- or 6-membered nitrogen containing heterocyclic aryl group, N-sulfonylated amino, amidoximo, N-aminoguanidino, amidino, guanidino, acylguanidino, cyclic derivatives of amidines and guanidines, acylated sulfonamide, a 5 or 6 membered alicyclic ring containing a basic nitrogen and optionally one or more oxygen atoms or CONR²R³ where at least one of R² and R³ contains an alkyl chain containing one or more basic nitrogen atoms and optionally oxygen or taken together form a 5- or 6- membered ring containing at least one basic nitrogen.
Y is carbon or nitrogen;
E is nothing when Y is nitrogen, and is hydrogen or halogen when Y is carbon;
G is hydrogen or halogen;
p is from 0 to 3, preferably 0;
provided at least one X includes a water solubilizing group as defined above or a nitrogen containing heterocycle;
and pharmaceutically acceptable salts thereof.

One of skill in the art can readily determine if a drug is an A2AR pathway agonist. For example, one may use known techniques to compare the binding of a radiolabeled putative A2AR agonist to a cell membrane that has A2AR, in the presence of known and unlabelled A2AR agonist. Alternatively, one may use published methods for evaluating effects of a putative A2AR agonist as competitor against a known radiolabeled A2A agonist, for binding to cell membranes that have A2AR. In addition, one may use known techniques to compare the effect of the putative A2AR agonist on cAMP accumulation in A2AR-expressing cells to the effect of a known A2AR agonist on cAMP accumulation in the same type of cell.

### Exemplary Drug Combinations

In some embodiments, an A2AR pathway agonist is co-administered with a therapeutic agent that causes weight gain. If a patient requires treatment with a drug having weight gain as a side effect (for example, certain diabetes treatments and certain antipsychotic drugs), an A2AR pathway agonist may be conjointly administered to prevent or limit the weight gain. In some aspects, a therapeutic agent that causes weight gain is one that induces at least 5 pounds of weight gain in at least a sub-population of patients receiving the agent, compared to an untreated control group.

Examples of anti-psychotic agents that may cause weight gain include clozapine, olanzapine, quetiapine, risperidone, ziprasidone, aripiprazole, trifluoperazine, flupenthixol, loxapine, perphenazine, chlorpromazine, haloperidol, fluphenazine decanoate, thioridazine, and pharmaceutically acceptable salts thereof.

An A2AR pathway agonist may be administered in combination with one or more anti-diabetic therapeutic. An anti-diabetic therapeutic is a therapeutic that is designed to lessen or limit the progression of at least one symptom of diabetes, for example elevated resting blood sugar levels. Exemplary anti-diabetic therapeutics include, for example, an aldose reductase inhibitor, a glycogen phosphorylase inhibitor, a sorbitol dehydrogenase inhibitor, a protein tyrosine phosphatase 1B inhibitor, a dipeptidyl protease inhibitor, insulin (including orally bioavailable insulin preparations), an insulin mimetic, metformin, acarbose, a peroxisome proliferator-activated receptor-γ (PPAR-γ) ligand such as troglitazone, rosaglitazone, pioglitazone or GW-1929, a sulfonylurea, glipazide, glyburide, or chlorpropamide wherein the amounts of the first and second compounds result in a therapeutic effect. Other anti-diabetic agents include a glucosidase inhibitor, a glucagon-like peptide-1 (GLP-1), insulin, a PPAR α/γ dual agonist, a meglitimide and an αP2 inhibitor. In an exemplary embodiment, an anti-diabetic agent may be a dipeptidyl peptidase IV (DP-IV or DPP-IV) inhibitor, such as, for example LAF237 from Novartis (NVP DPP728; 1-[[[2-[(5-cyanopyridin-2-yl)amino]ethyl]amino]acetyl]-2-cyano-(S)-pyrrolidine) or MK-04301 from Merck (see e.g., Hughes et al., Biochemistry 38: 11597-603 (1999)).

In some embodiments, an A2AR pathway agonist is co-administered with a medication that causes weight gain. Such co-administration can reduce undesirable side effects. Examples of medications that may cause weight gain, include for example, certain diabetes therapies, including, for example, sulfonylureas (such as glipizide, glyburide, and glimepiride), thiazolidinediones (such as pioglitazone and rosiglitazone), meglitinides, nateglinide, repaglinide, sulphonylurea medicines, and insulin; antidepressants, including, for example, tricyclic antidepressants (such as amitriptyline and imipramine), irreversible monoamine oxidase inhibitors (MAOIs), selective serotonin reuptake inhibitors (SSRIs), bupropion, paroxetine, and mirtazapine; steroids, such as, for example, prednisone; hormone therapy; lithium carbonate; valproic acid; carbamazepine; chlorpromazine; thiothixene; beta blockers (such as propranolol); alpha blockers (such as clonidine, prazosin and terazosin); and contraceptives including oral contraceptives (birth control pills) or other contraceptives containing estrogen and/or progesterone (Depo-Provera, Norplant, Ortho), testosterone or Megestrol. In another exemplary embodiment, A2AR pathway agonists may be administered as part of a smoking cessation program to prevent weight gain.

In another embodiment, a patient who is overweight or obese might be prescribed an A2AR pathway agonist as part of a weight-loss therapy, in combination with a drug or other therapy that causes weight loss or a body weight management agent. Body weight management agents are agents that prevent or limit weight gain, or cause weight loss. They may treat or prevent obesity. Examples of body weight management agents include appetite suppressants, fat absorption inhibitors, metabolic enhancers, fat mobilizers, and glycemic control agents.

Appetite suppressants are drugs that reduce sensations of hunger and/or increase satiety, and therefore cause a patient to ingest less food. These drugs typically act on the noradrenergic and serotoninergic neurotransmitter pathways. Phentermine is an example of an appetite suppressant that inhibits noradrenaline re-uptake, while sibutramine inhibits both scrotonin and noradrenaline re-uptake. Sibutraminc may be administered repeatedly over a long-term course of therapy. The formation definition of satiation is the point at which an individual becomes full or sated during an isolated eating episode. Similarly, satiety is defined as the period during which an individual remains sated after the ingestion of a prescribed amount of food.

Leptin is, inter alia, an appetite suppressant (McDuffie et al., Effects of Exogenous Leptin on Satiety and Satiation in Patients with Lipodystrophy and Leptin Insufficiency, The Journal of Clinical Endocrinology & Metabolism Vol. 89, No. 9 4258-4263). Leptin may be administered as a polypeptide or fragment or mimetic thereof; or as a nucleic acid encoding leptin, for example using gene therapy techniques.

Fat absorption inhibitors reduce a patient's energy intake without necessarily affecting the amount of food ingested. For example, fat absorption inhibitors may reduce the amount of fat allowed to pass through the gastrointestinal tract into the bloodstream. Orlistat, a fat absorption inhibitor, inhibits pancreatic lipase; activity of this enzyme is necessary for fat absorption.

Metabolic enhancers include drugs that increase thermogenesis without the need for an increase in physical activity. One example of a metabolic enhancer is a PKA activator. Mice that exhibit chronic stimulation of the protein kinase A (PKA) gene are lean and resistant to diet-induced obesity, suggesting that PKA agonists may stimulate energy expenditure and fat mobilization. (Cummings D E et al., 1996. “Genetically lean mice result from targeted disruption of the RII beta subunit of protein kinase A.” Nature 382: 622-626.) In addition, sirtuin activators such as resveratrol enhance metabolism and cause weight loss (for example, see US Patent Application No. 2006/0276416). In some instances, a sirtuin activator is a SIRT1 activator.

Fat mobilizers are drugs that act peripherally to reduce fat mass and/or decrease triglyceride synthesis. Leptin, leptin analogs, and leptin mimetics are fat mobilizers (in addition to being appetite suppressants). Patients with homozygous mutations in the leptin gene are one group of patients that benefit from leptin administration. Examples of leptin mimetics are provided in U.S. Pat. No. 5,756,461. In some embodiments, leptin is bound to apolipoprotein J.

Glycemic control agents are agents that promote normal blood sugar levels, and include diabetes treatments. Appropriate glycemic control agents include metformin and insulin; other examples are listed above. Some glycemic control agents promote weight gain and some do not, and the skilled practitioner will be able to select the appropriate glycemic control agent to prescribe to a given patient with this information in mind.

In some embodiments, the A2AR pathway agonist is co-administered with an anti-obesity agent, in some instances causing synergistic effects. Exemplary anti-obesity agents include, for example, phenylpropanolamine, ephedrine, pseudoephedrine, phentermine, a cholecystokinin-A agonist, a monoamine reuptake inhibitor (such as sibutramine), a sympathomimetic agent, a serotonergic agent (such as dexfenfluramine or fenfluramine), a dopamine agonist (such as bromocriptine), a melanocyte-stimulating hormone receptor agonist or mimetic, a melanocyte-stimulating hormone analog, a cannabinoid receptor antagonist, a melanin concentrating hormone antagonist, the OB protein (leptin), a leptin analog, a leptin receptor agonist, a galanin antagonist or a GI lipase inhibitor or decreaser (such as orlistat). Other anorectic agents include bombesin agonists, dehydroepiandrosterone or analogs thereof, glucocorticoid receptor agonists and antagonists, orexin receptor antagonists, urocortin binding protein antagonists, agonists of the glucagon-like peptide-1 receptor such as Exendin, and ciliary neurotrophic factors such as Axokine.

In another embodiment, an A2AR pathway agonist may be administered as a sleeping pill. In these embodiments, it may be conjointly administered with another medication that promotes sleep. General categories of sleep-promoting medications include certain antidepressants, barbiturates, benzodiazepines, typical antipsychotics (“major tranquilizers”), atypical antipsychotics, herbal sedatives, nonbenzodiazepine sedatives, as well as certain uncategorized sedatives.

Examples of sleep-inducing antidepressants include mirtazapine (Remeron®) and trazodone (Desyrel®). Barbiturates include amobarbital (Amytal®), pentobarbital (Nembutal®), and secobarbital (Seconal®). Benzodiazepines (“minor tranquilizers”) include alprazolam (Xanax®), bromazepam (Lexotan®), clonazepam (Klonopin®), diazepam (Valium®), estazolam (Prosom®), flunitrazepam (Rohypnol®), lorazepam (Ativan®), midazolam (Versed®), nitrazepam (Mogadon®), oxazepam (Serax®), triazolam (Halcion®), temazepam (Restoril®, Normison®, Planum®, Tenox® and Temaze®), and chlordiazepoxide (Librium®). Typical antipsychotics (“major tranquilizers”) include chlorpromazine (Thorazine®, Largactil®), fluphenazine (Prolixin®), haloperidol (Haldol®), loxapine succinate (Loxitane®), perphenazine (Etrafon®, Trilafon®), prochlorperazine (Compazine®), thiothixene (Navane®), trifluoperazine (Stelazine®, Trifluoperaz®), and zuclopentixol (Cisordinol®). Atypical antipsychotics include clozapine (Clozaril®), olanzapine (Zyprexa®), quetiapine (Scroquel®), risperidone (Risperdal®), and ziprasidone (Geodon®). Herbal sedatives include ashwagandha, kava (Piper methysticum), and valerian. Nonbenzodiazepine sedatives include eszopiclone (Lunesta®), zaleplon (Sonata®), zolpidem (Ambien®), and zopiclone (Imovane®, Zimovane®). Uncategorized sedatives include ethchlorvynol (Placidyl®), glutethimide (Doriden®), ketamine (Ketalar®, Ketaset®), methaqualone (Sopor®, Quaalude®), methyprylon (Noludar®), and ramelteon (Rozerem®).

Alternatively, an A2AR pathway agonist may be administered as a non-drowsy formulation. For example, some A2AR pathway agonists do not cause drowsiness. Other A2AR pathway agonists may cause drowsiness, but may be co-administered with a stimulant to prevent drowsiness. Categories of stimulants include phenethylamines, eugeroics, and NDRIs.

Phenethylamines include catecholamines (for example, dopamine, epinephrine, and norepinephrine) plant alkaloids (for example, ephedrine, pseudoephedrine, cathinone, and cathine), amphetamines and substituted amphetamines (for example, amphetamine, dextrorotatory isomer dextromethamphetamine), methylphenidate, certain bronchodilators (for example, albuterol and clenbuterol), and cyclopentamine. Another class of stimulants is norepinephrine and dopamine reuptake inhibitors (NDRIs), such as the antidepressant bupropion (Wellbutrin), pyrovalerone, mazindol and pipradrol.

Recently, there have been improvements in the area of stimulant pharmacology, producing a class of chemicals known as Ampakines, or eugeroics (good arousal). These stimulants tend to increase alertness without the peripheral (body) effects or addiction/tolerance/abuse potential of the traditional stimulants. They generally have minimal effect on sleep structure, and do not cause rebound hypersomnolence or “come down” effects. Currently, there are two stimulants in this class being used: modafinil and adrafinil, marketed as Provigil and Olmifon, respectively. Newer ampakines such as ampalex and CX717 have been developed but are still in clinical trials and have not yet been sold commercially. Another compound with similar effects to these drugs is Carphedon, which is sold as a general stimulant in Russia under the brand name Phenotropil.

### Exemplary Combination Therapies

In some instances, administration of an A2AR pathway agonist is combined with an additional therapy. The additional therapy may stimulate weight loss, or may prevent or limit weight gain. This therapy may be, for instance, surgery, a diet regimen, or an additional therapeutic agent.

One type of surgery for obesity is a restriction operation. Restriction operations for obesity include gastric banding and vertical banded gastroplasty, which exclusively restrict food intake.

A Roux-en-Y gastric bypass (RGB) is a gastric bypass procedure in which a stomach pouch is created at the top of the stomach. This may be done by stapling or vertical banding, and restricts food intake. Next, a portion of the small intestine is attached to the pouch to allow food to bypass the duodenum as well as the first portion of the jejunum. Reduced calorie and nutrient absorption result. In contrast, an extensive gastric bypass (biliopancreatic diversion) is a complicated gastric bypass operation. In this operation, portions of the stomach are removed. The remaining small pouch is connected directly to the final segment of the small intestine, completely bypassing both the duodenum and jejunum. Typically, gastric bypass operations are more effective than restriction operations. Gastric bypass operations generally result in the loss of two-thirds of a patient's excess weight within two years.

### Physiological Effects

In some embodiments, the A2AR pathway agonist results in overall decreased adipose content in a treated individual. In certain embodiments, the A2AR pathway agonist results in lower levels of one or more of: abdominal fat, subcutaneous fat, visceral fat, and epididymal fat.

Types of obesity that can be treated or prevented by administering an effective amount of an A2AR pathway agonist include, but are not limited to, android obesity, gynoid obesity, abdominal obesity, age-related obesity, diet-induced obesity, fat-induced obesity, hypothalamic obesity, morbid obesity, multigenic obesity, and visceral obesity.

Individuals that may be treated as described herein include eukaryotes, such as mammals, e.g., humans, ovines, bovines, equines, porcines, canines, felines, non-human primate, mice, and rats. Cells that may be treated include eukaryotic cells, e.g., from a subject described above, or plant cells, yeast cells and prokaryotic cells, e.g., bacterial cells. For example, an A2AR pathway agonist may be administered to farm animals to reduce their fat content to produce a more lean grade of meat.

Additional diseases or conditions that may be treated using the compositions disclosed herein include diabetes, sexual dysfunction, atherosclerosis, hypertension, insulin resistance, impaired glucose tolerance, hypercholesterolemia, hypertrigylceridemia, bulimia, lipodystrophies, hypertriglyceridemia, accumulation of intramyocellular lipid, hepatomegaly and hepatic steatosis, disordered glucose metabolism, hyperphagia, and thermodysregulation.

### Dosage Amounts and Timing of Administration

The amount of the compound, or an active salt or derivative thereof, required for use in treatment will vary not only with the particular salt or compound selected but also with the route of administration, the nature of the condition being treated and the age and condition of the patient and will be ultimately at the discretion of the attendant physician or clinician.

Additionally, the optimal concentration and/or quantities or amounts of any particular salt or composition may be adjusted to accommodate variations in the treatment parameters. Such treatment parameters include the clinical use to which the preparation is put, e.g., the site treated, the type of patient, e.g., human or non-human, adult or child, and the nature of the disease or condition.

The concentration and/or amount of any composition may be readily identified by routine screening in animals, e.g., rats, by screening a range of concentration and/or amounts of the material in question using appropriate assays. Known methods are also available to assay local tissue concentrations, diffusion rates of the salts or compositions, and local blood flow before and after administration of therapeutic formulations disclosed herein. One such method is microdialysis, as reviewed by T. E. Robinson et al., 1991, Microdialysis in the Neurosciences, Techniques, volume 7, Chapter 1. The methods reviewed by Robinson may be applied, in brief, as follows. A microdialysis loop is placed in situ in a test animal. Dialysis fluid is pumped through the loop. When salts or compositions such as those disclosed herein are injected adjacent to the loop, released drugs are collected in the dialysate in proportion to their local tissue concentrations. The progress of diffusion of the salts or compositions may be determined thereby with suitable calibration procedures using known concentrations of salts or compositions. In the art there are animal model systems for obesity and obesity-related diseases. for example, the diet-induced obesity (DIO) mouse model or a leptin-mutant mouse may be used. Once the correct dosage has been determined in a model system, the correct dose for humans may readily be determined according to Table A:

TABLE A
Conversion of Animal Doses to Human Equivalent Doses (HED) Based on
Body Surface Area (see e.g., Guidance for Industry Reviewers: Estimating
the Safe Starting Dose in Clinical Trials for Therapeutics in Adult Healthy
Volunteers, on the world wide web at
fda.gov/ohrms/dockets/98fr/02d-0492-gdl0001-vol1.pdf).

| | | To convert animal dose | |
|---|---|---|---|
| | | in mg/kg to HED^{a} in | |
| | To convert animal | mg/kg, either: | |
| | dose in mg/kg to | | Multiply |
| | dose in mg/m², | Divide | animal |
| | multiple by km | animal dose | dose |
| Species | below: | by: | by: |
| Human | 37 | — | — |
| Human Child (20 kg) | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea Pig | 8 | 4.6 | 0.22 |
| Rabbit | 12 | 3.1 | 0.32 |
| Dog | 20 | 1.8 | 0.54 |
| Monkeys^{b} | 12 | 3.1 | 0.32 |
| Marmoset | 6 | 6.2 | 0.16 |
| Squirrel Monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, human equivalent dose can be calculated from the formula: HED = animal dose in mg/kg × (animal weight in kg/human weight in kg)^{0.33}.
^{b}For example, cynomolgus, rhesus, stumptail.

In certain embodiments, the dosage of the subject salts and compositions provided herein may be determined by reference to the plasma concentrations of the therapeutic composition or other encapsulated materials. For example, the maximum plasma concentration (Cₘₐₓ) and the area under the plasma concentration-time curve from time 0 to infinity may be used.

In general, however, a suitable dose will be in the range of from about 0.5 to about 100 μg/kg, e.g., from about 10 to about 75 μg/kg of body weight per day, such as 3 to about 50 μg per kilogram body weight of the recipient per day, preferably in the range of 6 to 90 μg/kg/day, most preferably in the range of 15 to 60 μg/kg/day.

The compound is conveniently administered in unit dosage form; for example, containing 5 to 1000 μg, conveniently 10 to 750 μg, most conveniently, 50 to 500 μg of active ingredient per unit dosage form.

In certain embodiments, the active ingredient should be administered to achieve peak plasma concentrations of the active compound of from about 0.1 to about 10 nM, preferably, about 0.2 to 10 nM, most preferably, about 0.5 to about 5 nM.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple oral tablets.

In certain embodiments, the A2AR pathway agonists are administered over an extended period of time. For example, these agonists may be administered for 1 month, 3 months, 6 months, 1 year, 2 years, or 5 or more years. During this time the agonists may be administered repeatedly such as twice per day, once per day, once every two days, or once per week. In an alternative embodiment, the A2AR pathway agonist may be administered continuously in a sustained-release dosage form, such as an implanted capsule that releases the agonist over the course of months or years.

### Pharmaceutical Compositions

This application also discloses a pharmaceutical composition comprising an A2AR pathway agonist and a medicine that causes weight gain. The pharmaceutical composition may be formulated for systemic or local administration. The pharmaceutical composition may be formulated for oral administration, injection, subdermal administration, or transdermal administration. The pharmaceutical composition may further comprise at least one of a pharmaceutically acceptable stabilizer, diluent, surfactant, filler, binder, and lubricant.

The A2AR pathway agonist-containing compositions provided by this application may be administered to a subject in need of treatment by a variety of conventional routes of administration, including orally, topically, parenterally, e.g., intravenously, subcutaneously or intramedullary. Further, the compositions may be administered intranasally, as a rectal suppository, or using a “flash” formulation, i.e., allowing the medication to dissolve in the mouth without the need to use water. Furthermore, the compositions may be administered to a subject in need of treatment by controlled release dosage forms, site specific drug delivery, transdermal drug delivery, patch (active/passive) mediated drug delivery, by stereotactic injection, or in nanoparticles.

The A2AR pathway agonist-containing compositions may be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents. The pharmaceutical compositions formed by combining the compositions and the pharmaceutically acceptable carriers, vehicles or diluents are then readily administered in a variety of dosage forms such as tablets, capsules, granules, powders, lozenges, syrups, injectable solutions and the like. These pharmaceutical compositions can, if desired, contain additional ingredients such as flavorings, binders, excipients and the like. Thus, for purposes of oral administration, tablets containing various excipients such as L-arginine, sodium citrate, calcium carbonate and calcium phosphate may be employed along with various disintegrates such as starch, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in soft and hard filled gelatin capsules. Appropriate materials for this include lactose or milk sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof. The A2AR pathway agonist-containing compositions may also comprise a corrigent, a solubilizing agent, a suspension aid, or a coating agent.

For parenteral administration, solutions of the A2AR pathway agonist-containing compositions may be prepared in sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

The formulations, for instance tablets, may contain e.g. 3 to 800, or 20 to 600, e.g. 50, 250, 300, or 400, mg of the compositions disclosed herein, for instance A2AR pathway agonists.

Topical administration of the A2AR pathway agonist-containing compositions may also be indicated, for example, where the patient is suffering from gastrointestinal disorder that prevent oral administration, or whenever the medication is best applied to the surface of a tissue or organ as determined by the attending physician. Localized administration may also be indicated, for example, when a high dose is desired at the target tissue or organ. For instance, the A2AR pathway agonist may be delivered to adipose tissue. For example, a capsule designed for sustained release of an A2AR pathway agonist may be implanted in adipose tissue. Alternatively, the A2AR pathway agonist may be encapsulated in poly(lactide-co-glycolide) microspheres and injected into the individual. This targeting method is described in Richardson T P et al., (“Selective adipose tissue ablation by localized, sustained drug delivery”, Plast Reconstr Surg. 2003 Jul; 112(1): 162-70.) Alternatively, targeting peptides may be used to deliver the A2AR pathway agonists to adipose tissue. Exemplary targeting peptides, such as CKGGRAKDC, are described in (Kolonin M G et al., “Reversal of obesity by targeted ablation of adipose tissue.” Nat Med. 2004 Jun; 10(6):581-2.). For buccal administration the active composition may take the form of tablets or lozenges formulated in a conventional manner.

For purposes of transdermal (e.g., topical) administration, dilute sterile, aqueous or partially aqueous solutions (usually in about 0.1% to 5% concentration), otherwise similar to the above parenteral solutions, may be prepared.

Methods of preparing various pharmaceutical compositions with a certain amount of one or more salts or other active agents are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

In addition, in certain embodiments, the A2AR pathway agonist compositions may be lyophilized or subjected to another appropriate drying technique such as spray drying.

Formulations useful in the methods provided herein include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of a subject composition which may be combined with a carrier material to produce a single dose may vary depending upon the subject being treated, and the particular mode of administration.

Methods of preparing these formulations or compositions include the step of bringing into association A2AR pathway agonist-containing compositions with the carrier and, optionally, one or more accessory ingredients. In general, the formulations are prepared by uniformly and intimately bringing into association a subject composition with liquid carriers, or finely divided solid carriers, or both, and then, if necessary, shaping the product.

The salts and compositions described herein may be administered in inhalant or aerosol formulations. The inhalant or aerosol formulations may comprise one or more agents, such as adjuvants, diagnostic agents, imaging agents, or therapeutic agents useful in inhalation therapy. The final aerosol formulation may for example contain 0.005-90% w/w, for instance 0.005-50%, 0.005-5% w/w, or 0.01-1.0% w/w, of medicament relative to the total weight of the formulation.

It is desirable, but by no means required, that the formulations herein contain no components which may provoke the degradation of stratospheric ozone. In particular it is desirable that the formulations are substantially free of chlorofluorocarbons such as CCl₃F, CCl₂F₂ and CF₃CCl₃. As used to refer to ozone-damaging agents, “substantially free” means less than 1% w/w based upon the propellant system, in particular less than 0.5%, for example 0.1% or less.

The propellant may optionally contain an adjuvant having a higher polarity and/or a higher boiling point than the propellant. Polar adjuvants which may be used include (e.g., C₂₋₆) aliphatic alcohols and polyols such as ethanol, isopropanol and propylene glycol. In general, only small quantities of polar adjuvants (e.g., 0.05-3.0% w/w) may be required to improve the stability of the dispersion—the use of quantities in excess of 5% w/w may tend to dissolve the medicament. The formulations described herein may contain less than 1% w/w, e.g., about 0.1% w/w, of polar adjuvant. However, the formulations may be substantially free of polar adjuvants, such as ethanol. Suitable volatile adjuvants include saturated hydrocarbons such as propane, n-butane, isobutane, pentane and isopentane and alkyl ethers such as dimethyl ether. In general, up to 50% w/w of the propellant may comprise a volatile adjuvant, for example 1 to 30% w/w of a volatile saturated C₁-C₆ hydrocarbon.

Optionally, the aerosol formulations may further comprise one or more surfactants. The surfactants must be physiologically acceptable upon administration by inhalation. Within this category are included surfactants such as L-α-phosphatidylcholine (PC), 1,2-dipalmitoylphosphatidycholine (DPPC), oleic acid, sorbitan trioleate, sorbitan mono-oleate, sorbitan monolaurate, polyoxyethylene(20)sorbitan monolaurate, polyoxyethylene(20)sorbitan monooleate, natural lecithin, oleyl polyoxyethylene(2)ether, stearyl polyoxyethylene(2)ether, lauryl polyoxyethylene(4)ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil, and sunflower seed oil. Appropriate surfactants include lecithin, oleic acid, and sorbitan trioleate.

Ophthalmic formulations, eye ointments, powders, solutions and the like, are also contemplated as being within the scope of the disclosures herein.

Certain pharmaceutical compositions disclosed herein suitable for parenteral administration comprise one or more subject compositions in combination with one or more pharmaceutically acceptable sterile, isotonic, aqueous, or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition as an active ingredient. Subject compositions may also be administered as a bolus, electuary, or paste.

In solid dosage forms for oral administration (capsules, tablets, pills, dragees, powders, granules and the like), the subject composition is mixed with one or more pharmaceutically acceptable carriers and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using lactose or milk sugars, as well as high molecular weight polyethylene glycols and the like.

A tablet may be made by compression or molding, optionally with one or more accessory ingredients. Compressed tablets may be prepared using a binder (for example, gelatin or hydroxypropylmethyl cellulose), lubricant, inert diluent, preservative, disintegrant (for example, sodium starch glycolate or cross-linked sodium carboxymethyl cellulose), surface-altering or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

There has been widespread use of tablets since the latter part of the 19^{th} century and the majority of pharmaceutical dosage forms are marketed as tablets. Major reasons of tablet popularity as a dosage form arc simplicity, low cost and the speed of production. Other reasons include stability of drug product, convenience in packaging, shipping and dispensing. To the patient or consumer, tablets offer convenience of administration, ease of accurate dosage, compactness, portability, blandness of taste, ease of administration and elegant distinctive appearance.

Tablets may be plain, film or sugar coated, bisected, embossed, layered or sustained-release. They can be made in a variety of sizes, shapes and colors. Tablets may be swallowed, chewed or dissolved in the buccal cavity or beneath the tongue. They may be dissolved in water for local or topical application. Sterile tablets are normally used for parenteral solutions and for implantation beneath the skin

In addition to the active or therapeutic ingredients, tablets may contain a number of inert materials known as excipients. They may be classified according to the role they play in the final tablet. Examples of excipients include one or more of a filler, binder, lubricant and glidant. Other excipients which give physical characteristics to the finished tablet are coloring agents, and flavors (especially in the case of chewable tablets). Without excipients some drugs and pharmaceutical ingredients cannot be directly-compressed into tablets. This is primarily due to the poor flow and cohesive properties of most drugs. Typically, excipients are added to a formulation to impart good flow and compression characteristics to the material being compressed. Such properties are imparted through pretreatment steps, such as wet granulation, slugging, spray drying spheronization or crystallization.

Lubricants are typically added to prevent the tableting materials from sticking to punches, minimize friction during tablet compression, and allow for removal of the compressed tablet from the die. Such lubricants are commonly included in the final tablet mix in amounts usually of about 1% by weight.

Other desirable characteristics of excipients include the following: high-compressibility to allow strong tablets to be made at low compression forces; impart cohesive qualities to the powdered material; acceptable rate of disintegration; good flow properties that can improve the flow of other excipients in the formula; and cohesiveness (to prevent tablet from crumbling during processing, shipping and handling).

There are at least three commercially important processes for making compressed tablets: wet granulation, direct compression and dry granulation (slugging or roller compaction). The method of preparation and type of excipients are selected to give the tablet formulation the desired physical characteristics that allow for the rapid compression of the tablets. After compression, the tablets must have a number of additional attributes, such as appearance, hardness, disintegrating ability and an acceptable dissolution profile. Choice of fillers and other excipients will depend on the chemical and physical properties of the drug, behavior of the mixture during processing and the properties of the final tablets. Preformulation studies are done to determine the chemical and physical compatibility of the active component with proposed excipients.

The properties of the drug, its dosage forms and the economics of the operation will determine selection of the best process for tableting. Generally, both wet granulation and direct compression are used in developing a tablet.

One formulation comprises the following: an A2AR pathway agonist, and a binder. Examples of pharmaceutically acceptable binders include, but are not limited to, starches; celluloses and derivatives thereof, e.g., microcrystalline cellulose, hydroxypropyl cellulose hydroxylethyl cellulose and hydroxylpropylmethyl cellulose; sucrose; dextrose; corn syrup; polysaccharides; and gelatin. The binder, e.g., may be present in an amount from about 1% to about 40% by weight of the composition such as 1% to 30% or 1% to 25% or 1% to 20%.

Optionally, one, two, three or more diluents can be added to the A2AR pathway agonist formulations disclosed herein. Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 15% to about 40% by weight of the composition. In certain embodiments, diluents are microcrystalline cellulose which is manufactured by the controlled hydrolysis of alpha-cellulose, obtained as a pulp from fibrous plant materials, with dilute mineral acid solutions. Following hydrolysis, the hydrocellulose is purified by filtration and the aqueous slurry is spray dried to form dry, porous particles of a broad size distribution. Suitable microcrystalline cellulose will have an average particle size of from about 20 nm to about 200 nm. Microcrystalline cellulose is available from several suppliers. Suitable microcrystalline cellulose includes Avicel PH 101, Avicel PH 102, Avicel PH 103, Avicel PH 105 and Avicel PH 200, manufactured by FMC Corporation. The microcrystalline cellulose may be present in a tablet formulation in an amount of from about 25% to about 70% by weight. Another appropriate range of this material is from about 30% to about 35% by weight; yet another appropriate range of from about 30% to about 32% by weight. Another diluent is lactose. The lactose may be ground to have an average particle size of between about 50 μm and about 500 μm prior to formulating. The lactose may be present in the tablet formulation in an amount of from about 5% to about 40% by weight, and can be from about 18% to about 35% by weight, for example, can be from about 20% to about 25% by weight.

Optionally one, two, three or more disintegrants can be added to the A2AR pathway agonist formulations described herein. Examples of pharmaceutically acceptable disintegrants include, but are not limited to, starches; clays; celluloses; alginates; gums; cross-linked polymers, e.g., cross- linked polyvinyl pyrrolidone, cross-linked calcium carboxymethylcellulose and cross-linked sodium carboxymethylcellulose; soy polysaccharides; and guar gum. The disintegrant, e.g., may be present in an amount from about 2% to about 20%, e.g., from about 5% to about 10%, e.g., about 7% about by weight of the composition. A disintegrant is also an optional but useful component of the tablet formulation. Disintegrants are included to ensure that the tablet has an acceptable rate of disintegration. Typical disintegrants include starch derivatives and salts of carboxymethylcellulose. Sodium starch glycolate is one appropriate disintegrant for the A2AR pathway agonist formulations. In certain embodiments, the disintegrant is present in the tablet formulation in an amount of from about 0% to about 10% by weight, and can be from about 1% to about 4% by weight, for instance from about 1.5% to about 2.5% by weight.

Optionally one, two, three or more lubricants can be added to the A2AR pathway agonist formulations disclosed herein. Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant, e.g., may be present in an amount from about 0.1% to about 5% by weight of the composition; whereas, the glidant, e.g., may be present in an amount from about 0.1% to about 10% by weight. Lubricants are typically added to prevent the tableting materials from sticking to punches, minimize friction during tablet compression and allow for removal of the compressed tablet from the die. Such lubricants are commonly included in the final tablet mix in amounts usually less than 1% by weight. The lubricant component may be hydrophobic or hydrophilic. Examples of such lubricants include stearic acid, talc and magnesium stearate. Magnesium stearate reduces the friction between the die wall and tablet mix during the compression and ejection of the tablets. It helps prevent adhesion of tablets to the punches and dies. Magnesium stearate also aids in the flow of the powder in the hopper and into the die. It has a particle size range of 450-550 microns and a density range of 1.00-1.80 g/mL. It is stable and does not polymerize within the tableting mix. A lubricant such as magnesium stearate may also be employed in the formulations. In some aspects, the lubricant is present in the tablet formulation in an amount of from about 0.25% to about 6%; also appropriate is a level of about 0.5% to about 4% by weight; and from about 0.1% to about 2% by weight. Other possible lubricants include talc, polyethylene glycol, silica and hardened vegetable oils. In optional embodiments, the lubricant is not present in the formulation, but is sprayed onto the dies or the punches rather than being added directly to the formulation.

Other conventional solid fillers or carriers, such as, cornstarch, calcium phosphate, calcium sulfate, calcium stearate, magnesium stearate, stearic acid, glyceryl mono- and distearate, sorbitol, mannitol, gelatin, natural or synthetic gums, such as carboxymethyl cellulose, methyl cellulose, alginate, dextran, acacia gum, karaya gum, locust bean gum, tragacanth and the like, diluents, binders, lubricants, disintegrators, coloring and flavoring agents could optionally be employed.

Additional examples of useful excipients which can optionally be added to the composition are described in the Handbook of Pharmaceutical Excipients, 3rd Edition, edited by A. H. Kibbe, published by American Pharmaceutical Association, Washington D.C., ISBN: 0-917330-96-X, or Handbook of Pharmaceutical Excipients (4^{th} Edition), edited by Raymond C Rowe, published by Science and Practice.

Liquid dosage forms for oral administration include pharmaceutically acceptable emulsions, microemulsions, solutions, suspensions, syrups and elixirs. In addition to the A2AR pathway agonist-containing compositions, the liquid dosage forms may contain inert diluents, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, corn, peanut, sunflower, soybean, olive, castor, and sesame oils), glycerol, tetrahydrofuryl alcohol, polyethylene glycols and fatty acid esters of sorbitan, and mixtures thereof.

Suspensions, in addition to the A2AR pathway agonist-containing compositions, may contain suspending agents such as, for example, ethoxylated isostearyl alcohols, polyoxyethylene sorbitol, and sorbitan esters, microcrystalline cellulose, aluminum metahydroxide, bentonite, agar-agar and tragacanth, and mixtures thereof.

Dosage forms for transdermal administration include powders, sprays, ointments, pastes, creams, lotions, gels, solutions, patches, and inhalants. An A2AR pathway agonist-containing composition may be mixed under sterile conditions with a pharmaceutically acceptable carrier, and with any preservatives, buffers, or propellants that may be required. For transdermal administration, the complexes may include lipophilic and hydrophilic groups to achieve the desired water solubility and transport properties.

The ointments, pastes, creams and gels may contain, in addition to A2AR pathway agonist-containing compositions, other carriers, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays may contain, in addition to an A2AR pathway agonist-containing composition, excipients such as lactose, talc, silicic acid, aluminum hydroxide, calcium silicates and polyamide powder, or mixtures of such substances. Sprays may additionally contain customary propellants, such as chlorofluorohydrocarbons and volatile unsubstituted hydrocarbons, such as butane and propane.

Methods of delivering a composition or compositions via a transdermal patch are known in the art. Exemplary patches and methods of patch delivery are described in U.S. Pat. Nos. 6,974,588, 6,564,093, 6,312,716, 6,440,454, 6,267,983, 6,239,180, and 6,103,275.

In certain embodiments, a transdermal patch may comprise an outer backing foil, a matrix and a protective liner wherein a) the composition or compositions are present in the matrix in a solution (which may be oversaturated), b) the matrix may contain 1 to 5% activated SiO₂, and c) the matrix may have a moisture content of less than 0.7%. Moisture-free matrix patches which contain activated silicon dioxide in the matrix show an enhanced drug release into the skin.

In some embodiments, a transdermal patch may comprise: a substrate sheet comprising a composite film formed of a resin composition comprising 100 parts by weight of a polyvinyl chloride-polyurethane composite and 2-10 parts by weight of a styrene-ethylene-butylene-styrene copolymer, a first adhesive layer on the one side of the composite film, and a polyalkylene terephthalate film adhered to the one side of the composite film by means of the first adhesive layer, a primer layer which comprises a saturated polyester resin and is formed on the surface of the polyalkylene terephthalate film; and a second adhesive layer comprising a styrene-diene-styrene block copolymer containing a pharmaceutical agent layered on the primer layer. A method for the manufacture of the above-mentioned substrate sheet comprises preparing the above resin composition molding the resin composition into a composite film by a calendar process, and then adhering a polyalkylene terephthalate film on one side of the composite film by means of an adhesive layer thereby forming the substrate sheet, and forming a primer layer comprising a saturated polyester resin on the outer surface of the polyalkylene terephthalate film.

The A2AR pathway agonist-containing compositions herein can be packaged to produce a “reservoir type” transdermal patch with or without a rate-limiting patch membrane. The size of the patch and or the rate limiting membrane can be chosen to deliver the transdermal flux rates desired. Such a transdermal patch can consist of a polypropylene/polyester impervious backing member heat-sealed to a polypropylene porous/permeable membrane with a reservoir therebetween. The patch can include a pharmaceutically acceptable adhesive (such as a acrylate, silicone or rubber adhesive) on the membrane layer to adhere the patch to the skin of the host, e.g., a mammal such as a human. A release liner such as a polyester release liner can also be provided to cover the adhesive layer prior to application of the patch to the skin as is conventional in the art. This patch assembly can be packaged in an aluminum foil or other suitable pouch, again as is conventional in the art.

Alternatively, the A2AR pathway agonist-containing compositions herein can be formulated into a “matrix-type” transdermal patch. Drug Delivery Systems Characteristics and Biomedical Application, R. L Juliano, ed., Oxford University Press. N.Y. (1980); and Controlled Drug Delivery, Vol. I Basic Concepts, Stephen D. Bruck (1983) describe the theory and application of methods useful for transdermal delivery systems. The drug-matrix could be formed utilizing various polymers, e.g. silicone, polyvinyl alcohol. The “drug matrix” may then be packaged into an appropriate transdermal patch.

Another type of patch comprises incorporating the A2AR pathway agonist-containing composition directly in a pharmaceutically acceptable adhesive and laminating the drug-containing adhesive onto a suitable backing member, e.g. a polyester backing membrane. The drug should be present at a concentration which will not affect the adhesive properties, and at the same time deliver the required clinical dose.

Transdermal patches may be passive or active. Passive transdermal drug delivery systems currently available, such as the nicotine, estrogen and nitroglycerine patches, deliver small-molecule drugs. Many of the newly developed proteins and peptide drugs are too large to be delivered through passive transdermal patches and may be delivered using technology such as electrical assist (iontophoresis) for large-molecule drugs.

Iontophoresis is a technique employed for enhancing the flux of ionized substances through membranes by application of electric current. One example of an iontophoretic membrane is given in U.S. Pat. No. 5,080,646 to Theeuwes. The principal mechanisms by which iontophoresis enhances molecular transport across the skin are (a) repelling a charged ion from an electrode of the same charge, (b) electroosmosis, the convective movement of solvent that occurs through a charged pore in response the preferential passage of counter-ions when an electric field is applied or (c) increase skin permeability due to application of electrical current.

In some cases, it may be desirable to administer two pharmaceutical compositions separately to a patient. Therefore, the present application discloses, inter alia, a kit that comprises two separate pharmaceutical compositions: 1) an A2AR pathway agonist or prodrug thereof; and 2) a second pharmaceutical composition that induces weight gain, or prodrug thereof, or a pharmaceutically acceptable salt of either composition or prodrug. The kit may comprise a container for containing the separate compositions such as a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are widely used for the packaging of pharmaceutical unit dosage forms (tablets, capsules, and the like). Blister packs generally consist of a sheet of relatively stiff material covered with a foil of a plastic material that may be transparent. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. In some embodiments the strength of the sheet is such that the tablets or capsules can be removed from the blister pack by manually applying pressure on the recesses whereby an opening is formed in the sheet at the place of the recess. The tablet or capsule can then be removed via said opening

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2.sup.nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

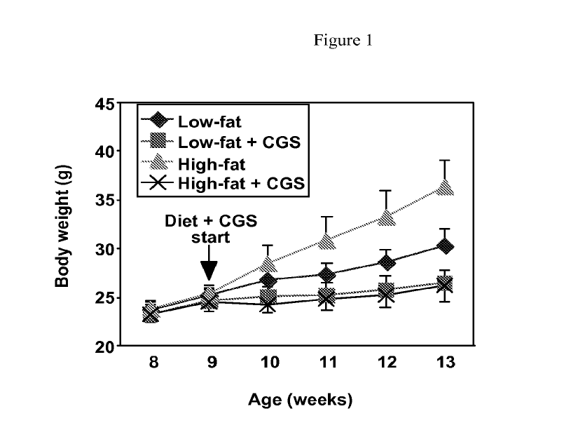

The mice used in this experiment were described in Ohta and Sitkovsky (Nature. 2001 Dec 20-27; 414(6866): 916-20). Mice were raised in a SPF (specific pathogen free) facility. The mice were fed a normal diet or a high-fat diet, but otherwise were raised in identical conditions. The mice were weighed at least 3 independent times each week, and averages of the 3 measurements were plotted. A subset of mice on either diet were treated with intraperitoneal injections of CGS 521680 (abbreviated CGS in FIG. 1) at a dosage of 0.5 mg/kg. Injections were administered once per day, during the day rather than at night. As a control, a second subset of mice on either diet was treated with vehicle only.

FIG. 1 is a chart depicting the weight of mice (y axis) versus age (x axis). Mice were either fed a low-fat or high-fat diet and were either treated or not treated with the selective A2AR agonist CGS21680. Data points marked with diamonds represent the weight of a mouse fed a low-fat diet and not treated with CGS21680. Data points marked with squares represent the weight of a mouse fed a low-fat diet and treated with CGS21680. Data points marked with triangles represent the weight of a mouse fed a high-fat diet and not treated with CGS21680. Data points marked with crosses represent the weight of a mouse fed a high-fat diet and treated with CGS21680.

### Example 2

The wild-type mice and A2AR-deficient mice used in this experiment were described in Ohta and Sitkovsky (Nature. 2001 Dec 20-27; 414(6866): 916-20). A2AR-deficient littermates or age-matched controls were raised in a SPF (specific pathogen free) facility. Age-matched and genetically matched experimental and control mice were sacrificed, and the epididymal fat was isolated by dissection.

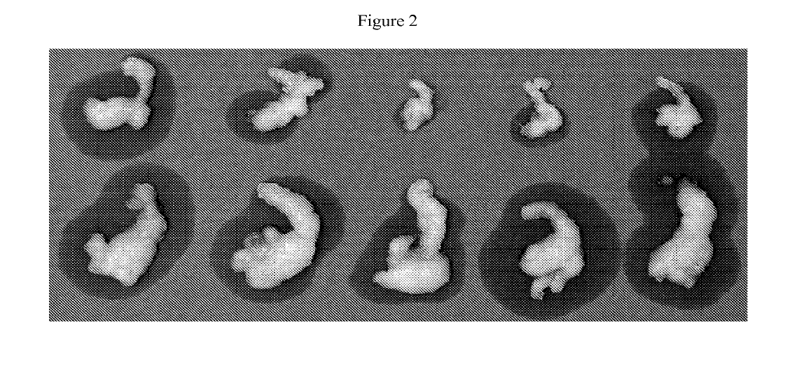

FIG. 2 illustrates the epididymal fat in mice deficient for A2AR. The epididymal fat is visible as a light-colored mass against a dark background. The top row depicts dissected epididymal fat from five different wild-type mice. The bottom row depicts dissected epididymal fat from five different age-matched mice in which A2AR was knocked out. The increased fat in the A2AR-deficient mice indicates that A2AR signaling promotes leanness, and loss of A2AR signaling promotes obesity.

### Example 3

Mice were untreated or treated with the A2AR agonist CGS21680 (Tocris) at different time points. CGS21680 (Tocris) was injected s.c. at a dose of 0.5 mg/kg daily either from week 9 to week 13, from week 13 to week 19, or from week 9 to week 19, as indicated in the FIG. 3 legends.

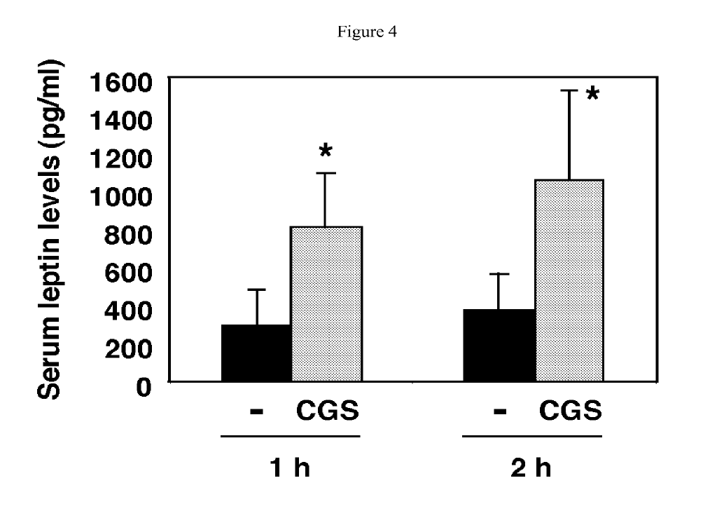

Male mice (8 mice per group) were fed a high-fat diet (where 60% of calories are derived from fat) or low-fat diet (where 10% of calories are derived from fat) and the weight of the mice was measured. Special diets were obtained from Research Diets (New Brunswick, N.J.). One group of mice received no A2AR agonist at all (represented by diamonds in the graphs of FIG. 4); another group of mice was received CGS21680 only from week 9 to 13 (triangles); another group of mice received CGS21680 only from week 13 to 19 (squares); another group of mice received CGS21680 from week 9 to 19 (crosses).

The left panel of FIG. 3 shows the weight of the mice given a high-fat diet. Some mice in the group that received no A2AR agonist at all (diamonds) almost doubled their weight between weeks 9 and 19, reaching up to 50 g total weight. Mice that were injected with A2AR agonist both from week 9 to 13 and from week 13 to 19 (crosses) gained little or no weight. The group of mice that were injected only from week 9 to 13 (triangles) gained essentially no weight between weeks 9 and 13, but afterwards (once A2AR agonist injections were discontinued) these mice started gaining weight. Mice that were not injected from week 9 to 13 (squares) reached almost 40 g of weight by week 13. Essentially no further weight gain was observed once these mice began receiving daily injections of the A2AR agonist.

The right panel of FIG. 3 shows the results of a low-fat diet in conjunction with A2AR agonist administration. The A2A receptor agonist prevented or limited weight gain even in mice on low fat diet. Mice in the group that received no A2AR agonist at all (diamonds) gained a small amount of weight reaching up to ˜35 g by week 19. This is much less than the weight reached by mice on the high-fat diet (˜50g). Mice that were injected with A2AR agonist both from week 9 to 13 and from week 13 to 19 (crosses) gained very little weight. The group of mice that received the A2A receptor antagonist only from week 9 to 13 (triangles) gained essentially no weight until week 13, but then (once A2AR agonist injections were discontinued) these mice started gaining weight. Mice that were not injected with from week 9 to 13 (squares) did gain weight by week 13. However, essentially no more weight gain was observed once these mice began receiving daily injections of A2AR agonist.

Comparing the mice treated continuously with CGS21680 (data marked with crosses in the left and right panels of FIG. 3), it can be seen that when the mice were treated with CGS21680, their body weight gain was minimal regardless of whether they received a high-fat or low-fat diet.

### Example 4

To elucidate the mechanism by which A2AR agonists reduce weight gain, serum leptin levels were quantified in mice treated with an A2AR agonist. 8 week old Female C57BL/6 mice received subcutaneous injection of the A2AR agonist CGS21680 (0.5 mg/kg). After 1 and 2 h, serum leptin levels were measured by ELISA. As shown in FIG. 4, administration of CGS21680 significantly increased serum leptin levels. The data shown in FIG. 4 represent average ±SD (n=4). The statistical significance was calculated by Student's t-test: *, p<0.05.

Since increased serum leptin levels correlate with reduced appetite, this experiment indicates that administration of A2AR pathway agonists reduces appetite.

### Example 5

Mice receiving a high-fat diet were treated with the A2AR agonists 2-[4-(2-aminoethylaminocarbonylethyl)phenylethylamino]-5′-N-ethylcarboxamidoadenosine (APEC) bistrifluoroacetic acid or 2 5′-N-ethylcarboxamidoadenosine (NECA). Male C57BL/6 mice (n=5) were provided a high-fat diet beginning at day 0. Also beginning on day 0, the mice were treated with daily subcutaneous injection of A2AR agonist. APEC was administered at 0.25 mg/kg/day, and NECA at 0.02 mg/kg/day.

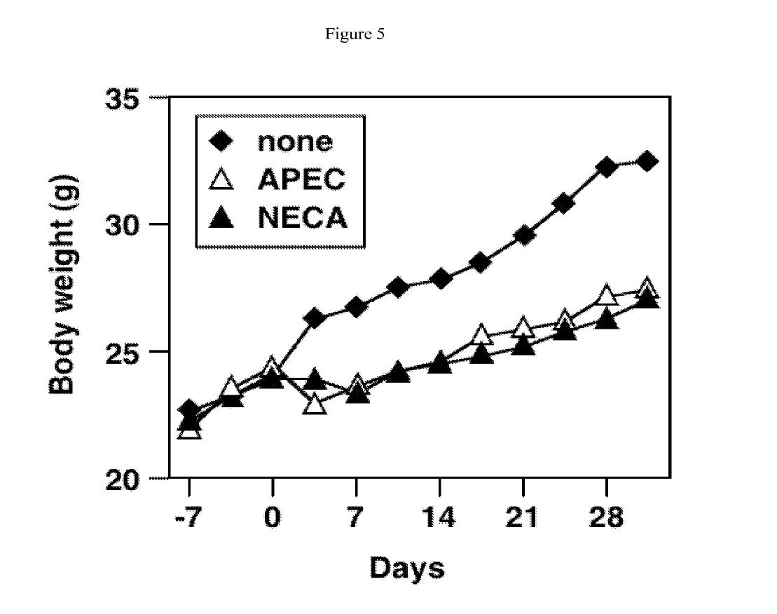

As FIG. 5 indicates, mice receiving APEC or NECA showed a dramatic reduction in weight gain compared to the control group. This experiment indicates that A2AR agonists with different chemical structures can reduce or prevent weight gain in animals consuming a high-fat diet.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

Thus while there have been described what are presently believed to be preferred embodiments of the invention, those skilled in the art will realize that changes and modifications may be made thereto without departing from the spirit of the invention, and it is intended to claim all such changes and modifications as fall within the true scope of the invention.

1. A method of reducing appetite, treating obesity, or inducing satiety, comprising administering a therapeutically effective amount of an A2AR pathway agonist to an animal in need thereof.

2. A method of preventing or limiting weight gain, comprising administering to an animal a therapeutically effective amount of an A2AR pathway agonist sufficient to reduce weight gain under conditions where the animal, in the absence of said agonist, would be susceptible to weight gain.

3. The method of claim 2, consisting of administering to an animal a therapeutically effective amount of an A2AR pathway agonist sufficient to reduce weight gain under conditions where the animal, in the absence of said agonist, would be susceptible to weight gain.

4. The method of claim 2, wherein the weight gain comprises a decrease of endogenous adenosine levels.

5. The method of claim 2, wherein the animal is not conjointly being treated with an antihistamine, a protein tyrosine phosphatase inhibitor, a COX-2 inhibitor, a FAAH inhibitor, a CRTH2 modulator, or an anti-cholinergic agent.

6. The method of claim 1 consisting of administering a therapeutically effective amount of an A2AR pathway agonist to an animal in need of treatment for obesity.

7. The method of claim 6, wherein the animal is not conjointly being treated with an antihistamine, a protein tyrosine phosphatase inhibitor, a COX-2 inhibitor, a FAAH inhibitor, a CRTH2 modulator, an anti-cholinergic agent, an adrenergic receptor antagonist, or a kinase inhibitor.

8. The method of claim 2 wherein the weight gain is associated with a therapeutic agent that induces weight gain, comprising administering a therapeutically effective amount of an A2AR pathway agonist to an animal that is being treated with the therapeutic agent.

9. The method of claim 8, consisting of administering a therapeutically effective amount of an A2AR pathway agonist to an animal that is being treated with the therapeutic agent.

10. The method of claim 8, wherein the weight gain that is prevented or limited comprises an increase in fat.

11. The method of claim 8, wherein the therapeutic agent that causes weight gain is a diabetes therapeutic.

12. The method of claim 11, wherein the diabetes therapeutic is at least one of: a sulfonylurea, a thiazolidinedione, a meglitinide, nateglinide, repaglinide, or insulin.

13. The method of claim 8, wherein the therapeutic agent that causes weight gain is an antidepressant.

14. The method of claim 13, wherein the antidepressant is at least one of: a tricyclic antidepressant, an irreversible monoamine oxidase inhibitor (MAOI), a selective serotonin reuptake inhibitor (SSRI), bupropion, paroxetine, or mirtazapine.

15. The method of claim 1, comprising conjointly administering to an animal in need thereof:
a. a therapeutically effective amount of an A2AR pathway agonist, and
b. one or more additional therapies, wherein the additional therapy treats, limits or prevents obesity,
wherein the animal is not being conjointly treated with an antihistamine, a protein tyrosine phosphatase inhibitor, a COX-2 inhibitor, a FAAH inhibitor, a CRTH2 modulator, an anti-cholinergic agent, an adrenergic receptor antagonist, or a kinase inhibitor.

16. The method of claim 15 consisting of conjointly administering to an animal in need thereof:
a. a therapeutically effective amount of an A2AR pathway agonist, and
b. one or more additional therapies, wherein the additional therapy treats, limits, or prevents obesity.

17. The method of claim 15, wherein the weight gain comprises an increase in fat.

18. The method of claim 15, wherein the additional therapy that treats, limits, or prevents obesity is the administration of a body weight management agent.

19. The method of claim 18, wherein the body weight management agent is an appetite suppressant.

20. The method of claim 19, wherein the appetite suppressant is selected from: aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, leptin, levamfetamine, levophacetoperane, mazindol, mefenorex, metamfepramone, methamphetamine, norpseudoephedrine, pentorex, phendimetrazine, phenmetrazine, phentennine, phenylpropanolamine, picilorex, and sibutramine.

21. The method of claim 18, wherein the body weight management agent is a fat absorption inhibitor.

22. The method of claim 21, wherein the fat absorption inhibitor is orlistat.

23. The method of claim 18, wherein the body weight management agent is a fat mobilization agent.

24. The method of claim 23, wherein the fat mobilization agent is leptin, a leptin analog, or a leptin mimetic.

25. The method of claim 15, wherein the additional therapy that treats, limits, or prevents obesity is a diet regimen, exercise regimen, or surgery.

26. The method of claim 25, wherein the surgery is gastric bypass surgery.

27. The method of claim 25, wherein the surgery is a restriction operation.

28. The method of claim 25, wherein the surgery is liposuction.

29. The method of claim 1 of inducing satiety in an animal, comprising administering a therapeutically effective amount of an A2AR pathway agonist to an animal in need thereof.

30. The method of claim 29, wherein the animal is suffering from bulimia.

31. The method of claim 30, comprising administering a therapeutically effective amount of an A2AR pathway agonist to an animal in need thereof.

32. The method of claim 1 wherein the animal is a human.

33. The method of claim 1 wherein the A2AR pathway agonist is a specific A2AR agonist.

34. The method of claim 33, wherein the A2AR agonist is a small molecule that binds A2AR.

35. The method of claim 33, wherein the A2AR agonist is APEC, ATL-146e, ATL202, ATL-313, ATL359, ATL844, ATL902, ATL908, ATL1222, ATL9844, binodenoson, CGS21680, CGS 22492C, CHA, CV-3146, CVT-3033, DMPA, GW328267X, LUF5835, MRE-0094, NECA, regadonoson, UK-371104, UK-432097, or CV1808.

36. The method of claim 1, wherein the A2AR pathway agonist is a compound of the formula: 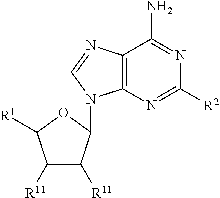
wherein:
R¹ is —C(O)NR³R⁴;
each R¹¹ is independently selected from —H, and OR⁵;
R⁵ is —H, C₁₋₄ alkyl, —C(O)C₁₋₄alkyl, or —C(O)H;
R² is selected from —H, and —NR⁶—C₁₋₄alkyl-phenyl-C₁₋₄alkyl wherein said alkyl groups are optionally substituted with —COOR⁷, or —CONR⁸R⁹;
R³, R⁴, R⁶, R⁷, R⁸, and R⁹ are each independently —H, —C₁₋₄alkyl, or —C₁₋₄alkyl-NH₂, or pharmaceutically acceptable salt thereof.

37. The method of claim 36, wherein R¹ is C(O)NHCH₂CH₃.

38. The method of claim 36, wherein R¹¹ is —OH.

39. The method of claim 36, wherein R² is —H or —NHCH₂CH₂-phenyl-CH₂CH₂—C(O)OH, or —NHCH₂CH₂-phenyl-CH₂CH₂—C(O)NH—CH₂—CH₂—NH₂.

40. The method of claim 1 wherein the A2AR pathway agonist reduces the activity of an inhibitor of the A2AR pathway.

41. The method of claim 40, wherein the inhibitor of the A2AR pathway is adenosine kinase or adenosine deaminase.

42. The method of claim 40, wherein the A2AR pathway agonist is a siRNA or ribozyme that reduces the levels of the inhibitor of the A2AR pathway.

43. The method of claim 1 wherein the A2AR pathway agonist is an activator of an adenosine synthesizing enzyme.

44. The method of claim 43, wherein the adenosine synthesizing enzyme is CD39 or CD73.

45. The method of claim 1 wherein the A2AR pathway agonist inhibits an enzyme that degrades adenosine.

46. The method of claim 45, wherein the A2AR pathway agonist is an inhibitor of adenosine kinase or adenosine deaminase.

47. The method of claim 1 wherein the A2AR pathway agonist is administered once nightly.

48. The method of claim 1 wherein the animal is a non-human animal.

49. The method of claim 1 wherein the animal is obese.

50. The method of claim 1 wherein the animal is non-obese.

51. The method of claim 1 wherein the A2AR pathway agonist promotes sleep.

52. The method of claim 1 wherein the A2AR pathway agonist does not induce drowsiness.

53. The method of claim 1 wherein the A2AR pathway agonist is administered once per day, prior to sleeping.

54. The method of claim 1 wherein the animal is suffering from insomnia.

55. The method of claim 1 wherein the animal is suffering from an inflammatory disease.
